(19) United States
(12) Patent Application Publication
(10) Pub. No.: US 2025/0057906 A1
(43) Pub. Date: Feb. 20, 2025

(54) BROCCOLI EXTRACT, NUTRACEUTICAL, AND METHODS OF TREATING INFLAMMATION AND CANCER

(71) Applicant: THE BOARD OF TRUSTEES OF THE UNIVERSITY OF ILLINOIS, URBANA, IL (US)

(72) Inventors: KATHERINE WARPEHA, CHICAGO, IL (US); DIVYA BIJUKUMAR, ROCKFORD, IL (US)

(73) Assignee: THE BOARD OF TRUSTEES OF THE UNIVERSITY OF ILLINOIS, URBANA, IL (US)

(21) Appl. No.: 18/805,952

(22) Filed: Aug. 15, 2024

Related U.S. Application Data
(60) Provisional application No. 63/533,076, filed on Aug. 16, 2023.

(51) Int. Cl. A61K 36/31; A61P 29/00

(52) U.S. Cl. CPC A61K 36/31; A61P 29/00; A61K 2236/19; A61K 2236/333; A61K 2236/51

(57) ABSTRACT

Disclosed are broccoli plant extracts and nutraceuticals including the same that have increased levels of anti-inflammatory chemicals. Also provided are exosomal preparations containing the broccoli plant extracts and nutraceuticals as well as methods of using the broccoli plant extracts and nutraceuticals for treating inflammation and treating or reducing the risk of cancer recurrence in a subject.

### INTRODUCTION

This application claims the benefit of priority from U.S. Provisional Application Ser. No. 63/533,076, filed Aug. 16, 2023, the contents of which are incorporated herein by reference in their entireties.

### STATEMENT REGARDING ELECTRONIC FILING OF A SEQUENCE LISTING

A Sequence Listing in XML format, entitled “UIC106US.XML,” 9, 801 bytes in size, generated Aug. 4, 2024, and filed herewith, is hereby incorporated by reference into the specification.

### BACKGROUND

The most widespread form of systemic inflammatory arthritis is rheumatoid arthritis. With a 2:1 female to male ratio, it affects about 1% of the population and the prevalence increases as the patient becomes older. non-steroidal anti-inflammatory drugs (NSAIDs), Disease-modifying antirheumatic drugs (DMARDs), and corticosteroids are some forms of the drugs that are currently being prescribed for the condition. However, these drugs are expensive and come with a number of side effects. Owing to such reasons, patients around the world have been turning to various natural products such as curcumin, L-sulforaphane (LSF and SF), and the like. LSF has been shown to provide a promising anti-inflammatory response in cancer. However, the bioavailability and stability of natural product in vivo are limited. Harsh processing associated with synthetic nanocarrier preparations can degrade the active molecules during preparation.

In Arabidopsis, a genetic model for plant metabolism, mutants have been identified that produce phenylpropanoids, which reduce plant cellular stress. Growth conditions have been refined for phenolic induction, then tested in stressed primary cultures derived from a mouse APOE gene replacement model of low, average, and high risk of developing Alzheimer's Disease (AD) (Ghura et al. (2016) Sci Rep. 6:29364). Data indicated that extracts reduced negative aspects of the inflammatory response, especially in the difficult-to-treat carriers of APOE4 alleles (Ghura et al. (2016) Sci Rep. 6:29364).

Broccoli is a common food plant that has been shown to produce isothiocyanates exhibiting cancer preventive activity (Zhang et al. (1994) Proc. Natl. Acad. Sci. USA 91:3147-50; Fahey et al. (1997) Proc. Natl. Acad. Sci. USA 94:10367-72; Zhang & Talalay (1994) Cancer Res. 54: 1976s-81s). Many plant types of the Brassicaceae have been indicated as part of an anti-inflammatory diet (Wagner et al. (2013) Oxid. Med. Cell. Longev. 2013:964539). However, broccoli in its typical form for food consumption is not optimized in its chemical constituents to target pain as desired chemicals in food grade broccoli are not induced, or are so low in bioactive chemical concentrations, that consuming excessive amounts a day would be required to achieve medicinal effects.

Accordingly, there is need for plants having an inducible, stable anti-inflammatory plant chemical profile that are non-genetically modified and provide extracts with increased bioavailability and effectiveness in alleviating pain.

### SUMMARY

The compositions and methods herein are based, in part, on the development of broccoli plants, growth conditions, and extraction procedures for enhanced levels of anti-inflammatory compounds. Accordingly, in one aspect is provided a broccoli extract comprising an enhanced level of anti-inflammatory compounds, the broccoli extract prepared by a method comprising (a) subjecting a broccoli plant to at least one abiotic stimulus; (b) extracting aerial material of the broccoli plant with 90% to 95% ethanol; (c) drying the extract; (d) reconstituting the extract in dimethylsulfoxide (DMSO) to form a concentrated broccoli extract, the broccoli extract being at a concentration of about 150 mg/mL; and (e) optionally treating the concentrated broccoli extract with an S9 liver microsome fraction, wherein the concentrated broccoli extract comprises at least 20 weight percent sinapates and at least 30 weight percent flavonoids. A nutraceutical product comprising the broccoli extract is also provided as is a nutraceutical product comprising the broccoli extract encapsulated in exosomes.

In some aspects, a nutraceutical broccoli product comprising an enhanced level of anti-inflammatory compounds is provided, the broccoli product prepared by a method comprising subjecting a broccoli plant to at least one abiotic stimulus to enhance levels of anti-inflammatory compounds in the broccoli plant and harvesting edible portions of the broccoli plant.

In other aspects, a method of treating inflammation in a subject in need thereof is provided, which comprises administering to the subject an effective amount of a broccoli extract, nutraceutical product, or nutraceutical broccoli product herein.

In another aspect, a method of treating or reducing the risk of cancer recurrence in a subject is provided, which comprises administering to the subject an effective amount of a broccoli extract, nutraceutical product, or nutraceutical broccoli product herein.

### BRIEF DESCRIPTION OF THE DRAWINGS

FIGS. 1A-1D show that original extract (OE) reduces inflammation in rabbit disc cells in vitro and increases collagen gene expression in vivo. FIGS. 1A-1C, Rabbit disc cells were pretreated with different concentrations of OE and then stimulated with lipopolysaccharide (LPS) for 16 hours. Gene expression of Interleukin-8 (IL-8; FIG. 1A) and nitric oxide synthase 2 (NOS2; FIG. 1B) was analyzed. Statistically significant differences between OE-treated and vehicle control (VC, saline) from LPS (P<0.05) are indicated (*). Conditioned media was collected to quantify interleukin-8 (IL-8) protein. FIG. 1D, Rabbits (n=8) underwent surgical disc injury and intradiscal treatment with OE or VC. After 3 weeks, intervertebral disc tissues were isolated for RNA analysis of phenotypic disc and inflammatory genes: collagen type I (COL1), collagen type II (COL2), chemokine C-C motif ligand (CCL) 2, CCL5 and IL-8. Cell culture experiment data were analyzed with One-way ANOVA with multiple comparisons and included in the significant difference (*) on graphs. In vivo rabbit disc gene expression was analyzed with multiple unpaired t-tests. Error bars represent SEM.

FIGS. 2A-2C show that phenylpropanoid-enriched broccoli extracts reduce pain behavior in mice. FIG. 2A, OE was administered as an i.p. (20 μL, 375 μg/mL) treatment 30 minutes before formalin injection to the paw. At time 0 minute, mice were injected in the rear left dorsal side of the paw with formalin as described in the Methods, and timing commenced for pain behavior (licking the injured paw); early (0-5 minutes) and late (40-55 minutes) are shown. For OE-treated mice, significantly reduced pain behavior in mice was apparent only during the early Phase I (0-5 minutes; P=0.0032 (n=8)) and was not significant in the later phase (Phase II; 40-55 minutes (shown on left)). VC is set to 1.00 for comparison of OE, and for the S9-treated OE. S9-treated OE (20 μL, 375 μg/mL) was significant for both early and late behavior as shown on the FIG. 2A (phase I P=0.0018; phase II P=0.0062). FIG. 2B, Pooled male and female mice pain behavior over the 0-70 minutes period indicated that reduction in pain (licking of injured foot) was significant with S9-treated OE but not OE. An unpaired t-test with Welch's correction resulted in a P-value of 0.0012 for S9 and 0.2096 for OE (n=8). FIG. 2C, Mice were injected at T=−30 minutes with OE. At zero, mice were allowed to explore in their activity maze as described in Methods. OE showed no significant changes in general mice activity, eliminating possible confounding variables in the assessments of pain reduction.

### DETAILED DESCRIPTION

Plant-derived phenylpropanoids can be potent cancer interventions in inflammatory-based diseases and therapy. Among the commonly tested phenylpropanoids, flavonoids are shown to target inflammatory pathways and cancer metabolism, and induce cancer cell apoptosis. In addition, polyphenols are shown to have a synergistic effect when combined with chemotherapeutics. Cruciferous plants such as broccoli are rich in anti-inflammatory chemicals. However, the bioavailability and stability of natural products in vivo are limited. Accordingly, methods are provided herein for producing broccoli extracts and nutraceutical compositions that exhibit an enhanced level of anti-inflammatory compounds. The extracts and nutraceutical compositions find application in treating inflammatory diseases and conditions, as well as in modulating cancer metabolism and inducing cancer cell apoptosis.

In one aspect, a broccoli extract composed of an enhanced level of anti-inflammatory compounds is provided, wherein the broccoli extract is prepared by a method including the steps of (a) subjecting a broccoli plant to at least one abiotic stimulus; (b) extracting aerial material of the broccoli plant with 90% to 95% ethanol; (c) drying the extract; (d) reconstituting the extract in dimethylsulfoxide (DMSO) to form a concentrated broccoli extract, the broccoli extract being at a concentration of about 150 mg/ml; and (e) optionally treating the concentrated broccoli extract with an S9 liver microsome fraction, wherein the concentrated broccoli extract comprises at least about 20 weight percent sinapates and at least about 30 weight percent flavonoids. The term “about” is used herein refers to a degree of deviation. It means approximately, in the region of, roughly, or around. When the term “about” is used in conjunction with a numerical range, it modifies that range by extending the boundaries above and below the numerical values set forth, e.g., ±0.1, ±0.5, ±1, ±5, or any value or range therebetween. It is understood that support in this specification for numerical values used in connection with the term “about” is also provided for the exact numerical value itself as though “about” were not used.

As used herein, an “extract” refers to those substances prepared using a solvent, e.g., ethanol, water, steam, superheated water, methanol, hexane, dichloromethane, chloroform liquid, liquid CO₂, liquid N₂, propane, supercritical CO₂, or any combination thereof. The term “extract” can also include fractionation products obtained by further fractionation of an extract. That is, the extract includes not only extracts obtained using the above-described extraction solvents but also filtrates and/or concentrates obtained by further purification of the extracts. The extract herein is also intended to include fractions obtained by or fractionation products through passing the extracts filtration membranes having a specific cut-off molecular weight, and fractions obtained through various additional purification processes, for example, various chromatography separation processes (those depending on size, charge, hydrophobicity, or affinity).

Extracts can refer to an extract in a liquid form or can refer to a product obtained from further processing of the liquid form, such as a dried powder or other solid form. Extracts may take many forms including, but not limited to, solid, liquid, particulate, chopped, distillate, etc. and may be performed by any number of procedures or protocols, such as chopping, grinding, pulverizing, boiling, steaming, soaking, steeping, infusing, applying a gas, etc., and may employ any suitable reagents, such as water, alcohol, steam, or other organic materials. In some embodiments, extracts can be phytoextracts made from specific parts of a source, such as the skin, pulp, leaves, flowers, of a plant etc., or can be made from the whole source.

In some embodiments, extracts and compositions (e.g., nutraceutical compositions) herein are prepared from a broccoli plant. A broccoli plant herein refers to a plant of the genus species Brassica oleracea var. italica. In some embodiments, the broccoli plant is grown from chemically mutated seeds. In one embodiment, the mutagenic chemical is ethylmethane sulfonate (EMS). In some embodiments, any or all edible or aerial parts of a broccoli plant are used in the methods, extracts, and compositions of this invention including, but not limited to, the stem, cotyledons, hypocotyls, inflorescence, leaves, or combination thereof. In some embodiments, the methods, extracts, and compositions of this invention use the stem of broccoli. In some embodiments, the methods, extracts, and compositions of this invention use the leaves of broccoli. In some embodiments, a broccoli extract is prepared by grinding aerial plant material (e.g., leaves) of a broccoli plant (e.g., a seedling) and sonicating the ground material in 90% to 95% ethanol, e.g., 90%, 91%, 92%, 93%, 94%, or 95% ethanol. In some embodiments, a broccoli extract is prepared by grinding aerial plant material of a broccoli plant (e.g., a seedling) and extracting the plant material with dichloromethane.

According to the methods, extracts, and compositions herein, prior to extraction a broccoli plant is subjected to at least one (e.g., 1, 2, 3, 4, 5 or more) abiotic stimulus. The step of applying at least one abiotic stimulus to a plant induces the expression of key enzymes and/or increases pools of enzyme substrates, which in turn leads to formation and accumulation of the desired compound or class of compounds, in some embodiments, induced or enhanced levels of anti-inflammatory compounds produced by the broccoli plant. Comparative terms such as “improved,” “enhanced,” and like terms can be used to state a result achieved or property present in a formulation or process that has a measurably better or more positive outcome than the thing to which comparison is made. In some instances comparison may be made to the prior art. In some embodiments, an induced or enhanced the level of anti-inflammatory compounds in a broccoli plant subjected to at least one abiotic stimulus may be compared to a broccoli plant grown under the same conditions and not subjected to at least one abiotic stimulus.

The broccoli plant may be subjected to at least one abiotic stimulus, harvested, and subsequently be used for the preparation of broccoli extracts and/or compositions (e.g., nutraceutical compositions). As used herein, the term “harvest” refers to the process or period in time in which a plant or plant part is removed from its natural environment. For example, a whole plant is harvested when it is removed from the soil in which it was planted, whereas a leaf is harvested when it is removed from the whole plant. In some embodiments, a broccoli seedling is subjected to at least one abiotic stimulus, e.g., a 4, 5, 6, 7, 8, 9, 10, 11, 12 day old, or more broccoli seedling. In some embodiments, the broccoli plant subjected to at least one abiotic stimulus has not flowered. In some embodiments, at least one abiotic stimulus is used herein. In some embodiments, more than one abiotic stimulus is used, e.g., 2, 3, 4, 5, 6, 7, 8, 9 or 10.

An “abiotic” stimulus is defined as a non-living factor (e.g., substance or condition) that has an impact on the gene expression or metabolism of a living organism. Examples of abiotic stimuli of use in the methods, extracts, and compositions herein include, but are not limited to, hyperosmotic stresses such as drought or high salt, temperature stresses such as cold or heat, aberrant nutrient conditions, mechanical shock, flooding, wounding, anaerobic stress, oxidative stress, ozone, light stress, heavy metals, toxic chemicals, ultrasound, ultraviolet light, elicitor chitosan treatment, modified lecithin treatment, abscisic acid treatment, false germination and combinations thereof.

Hyperosmotic stimuli include exposure to drought, high salt or high solute conditions. Whereas drought can be achieved by reducing or eliminating the amount of water a plant receives, a high salt or hyperosmotic condition can include exposing a plant to a solution containing, e.g., at least 150 mM NaCl or at least 300 mM mannitol. A plant can be flooded or waterlogged by covering or submerging the plant in water.

Temperature stress includes exposure to either high or low temperature. Low temperature or freezing stress may be conditions in which the average temperature of the plant environment is 15° C. or lower, more severely 10° C. or lower, and still more severely 5° C. or lower, e.g., 5° C., 4° C., 3° C., 2° C., or 1° C. High temperature stress may be conditions in which an average temperature of the plant environment is 25° C. or higher, more severely 30° or higher, and still more severely 35° C. or higher. Temperature stress durations may range from about 10 minutes to about 3 hours, with preferred durations between 30 minutes and 1 hour.

Aberrant nutrient conditions refer to high or low nitrogen, phosphorus, iron and the like.

The term “anaerobic stress” means any reduction in oxygen levels sufficient to produce a stress as hereinbefore defined, including hypoxia and anoxia.

Oxidative stress refers to any stress, which increases the intracellular level of reactive oxygen species.

Wounding is the disturbance of the natural plant, tissue and/or cell structure by methods like cutting, slicing, abrasion, squashing, breaking, peeling, crushing, pressing, slashing, grinding, fluid injection, osmotic shock, detaching, shredding, rubbing, piercing, pinching and tearing.

Ultraviolet light has been reported to be an abiotic stimulus that induces an increase in phenolic compounds. UVB has been the most frequently used source of irradiation for increasing phenol antioxidant production in plants. The UVB spectral band (280-315 μm) contributes less than 2% of the short-wave photons in sunlight. Application of UVB irradiation at a fluence (dose) in the range of about 10⁴ μmol m⁻² to 10⁵ μmol m⁻², e.g., 10⁴ μmol m⁻² may be carried out by known methods. UVC treatment (100-280 nm) at a fluence (dose) in the range of about 10⁴ μmol m⁻² to 10⁵ μmol m⁻², e.g., 10⁴ μmol m⁻² may also be carried out by known methods. In some embodiments, plants are subjected to treatment with both UVB and UVC irradiation. In some embodiments, broccoli plants are subjected to treatment with sublethal UV treatments of 254 nm and 315 nm, each at a fluence (dose) of 10⁴ μmol m⁻². Irradiation durations depend on the UV intensity and in certain embodiments will range from about 10 minutes to about 3 hours, with preferred durations between 30 minutes and 1 hour. The durations and intensities may be determined using routine skill in the art and may vary depending on the commercial set up for handling large quantities of plant material. In some embodiments, irradiation is conducted at temperatures ranging from about 20-40° C.

Plant growth and development are impacted by properties of light, including photosynthetic photon flux density (PPFD or light intensity) defined as number of photosynthetically active radiation (PAR) photons received per unit time in unit area measured in umol·m⁻²·s⁻¹, daily light integral (DLI), the amount of PAR received by the plants in a day, and light quality, the spectral distribution of light (nm). PAR ranging from 400-700 nm, is the spectrum of light that plants use for photosynthesis and growth. Spectrum of light outside this range, including ultraviolet (UV, 100-400 nm) and far-red light (700-800 nm), may also influence vegetative or reproductive plant growth and the synthesis of specialized metabolites, dependent upon species. The red spectrum (600-700 nm) had the highest relative quantum yield of CO: assimilation compared to green (500-600 nm) and blue (400-500 nm) light, up to 500 μmol·m⁻²·s⁻¹ PPFD as observed in lettuce. However, at higher light intensities, green light had a similar relative quantum yield of CO: due to its ability to penetrate deeper into the leaf and excite the deeper chlorophyll. In contrast, a fraction of blue light gets absorbed by non-photosynthetic pigments, such as anthocyanins or photosynthetic carotenoids, resulting in lower photosynthetic efficiency than red spectrum. In some embodiments, broccoli plants are subjected to treatment with blue light at a fluence (dose) of 10⁴ μmol m⁻².

False germination or false malting describes a treatment similar or identical to malting techniques as practiced by a person skilled in the art. However, as the seeds are in dormancy, for example in secondary dormancy, the seeds subjected to false malting do not germinate. See U.S. Pat. No. 10,334,689 B2, incorporated herein by reference.

In some embodiments, a broccoli plant (e.g., broccoli seedling) is grown under controlled sequences of light, darkness, and temperature. In some embodiments, a broccoli plant is treated with ultraviolet light, a temperature of about 4° C., darkness, and/or blue light. In some embodiments, a broccoli plant is treated with sublethal UV treatments of 254 nm and 315 nm, each at a fluence (dose) of 10⁴ μmol m⁻², followed by cold (1 hour at 4° C.) and blue light (10⁴ μmol m⁻²), with 1 minute between treatments on day 5 of growth and then subjected to darkness for about 12 hours prior to harvest.

In some embodiments, aerial broccoli plant material and/or extracts of the same may be dried (e.g., to remove solvent) using any conventional means including, but not limited to, rotary evaporation, freeze drying (lyophilization), or a combination thereof, to produce a dried powder, semi-dried powder, or other solid form. In some embodiments, the dried material is a dried broccoli extract, and the dried broccoli extract may be resuspended or reconstituted in a suitable solvent to form a concentrated broccoli extract. Examples of suitable solvents include, but are not limited to, ethanol, water, ethyl acetate, methanol, saline, DMSO, and combinations thereof. In some embodiments, the dried broccoli extract is reconstituted in DMSO to form a concentrated broccoli extract. In some embodiments, the concentrated broccoli extract has a concentration of about 1 mg to 1000 mg dried broccoli extract per mL solvent, e.g., 1, 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 mg/mL.

Extracts typically have a given purity percentage and can be relatively pure (e.g., 70 to 90%) to highly pure (e.g., 65 to 99.9%). In some embodiments, the purity of an extract can be controlled by, or be a function of the extraction process or protocol. In some embodiments, an extract may include one or more active fractions or active agents. In some embodiments, a broccoli extract (e.g., a dried or concentrated broccoli extract) herein comprises sinapates and/or flavonoids.

In some embodiments, a concentrated broccoli extract (e.g., an ethanol extract) comprising about 150 mg/mL broccoli extract includes at least 20 weight percent sinapates and at least 30 weight percent flavonoids. In other embodiments, a concentrated broccoli extract comprising about 150 mg/mL broccoli extract includes at least 30 weight percent sinapates. In other embodiments, a concentrated broccoli extract comprising about 150 mg/mL broccoli extract includes at least 40 weight percent sinapates. In some embodiments, a concentrated broccoli extract comprising about 150 mg/mL broccoli extract includes at least 40 weight percent flavonoids. In other embodiments, a concentrated broccoli extract comprising about 150 mg/mL broccoli extract includes at least 50 weight percent flavonoids. In other embodiments, a concentrated broccoli extract comprising about 150 mg/mL broccoli extract includes at least 50 weight percent flavonoids and optionally at least 30 weight percent sinapates. In other embodiments, a concentrated broccoli extract comprising about 150 mg/mL broccoli extract includes at least 50 weight percent flavonoids and optionally at least 40 weight percent sinapates.

In some embodiments, a concentrated broccoli extract (e.g., a dichloromethane extract) reconstituted to a 20% solution in methanol (e.g., 20 wt % dried broccoli extract) comprises at least 8% sulforaphane.

As used herein, “sinapates” refer to simple and more complex phenylpropanoids including sinapoyl malate, 1,2 di-O-sinapoyl-β-glucose, 1,2 di-O-sinapoyl-β-glucose, sinapoyl spermidine, sinapoyl hexoside, disinapoyl dihexoside, trisinapoyl dihexoside, and the like.

“Simple phenolics” means low molecular weight phenols having a single aromatic ring including cinnamic acid, trans-cinnamic acid, para-coumeric acid, and the like.

“Polyphenols” include phenylpropanoids comprising two or more aromatic rings, including flavonoids and other derived molecules.

“Polyamine” refers to low molecular weight diamines having two or more amino groups. Representative polyamines found in broccoli include spermine (N-(3-aminopropyl)-1,4-butane diamine), spermidine spermine (N,N-bis(3-aminopropyl)-1,4-butane diamine) and putrescine (1,4-butane diamine). Polyamines have been shown to exhibit antioxidant and anti-inflammatory activity.

“Flavonoids” refer to a family of polyphenolic plant compounds. Flavonoids comprise fifteen carbon atoms with two aromatic rings connected by a three-carbon bridge. Representative flavonoids found in broccoli include isorhamnetin, kaempferol, myricetin and quercetin. Flavonoids have been shown to exhibit anti-inflammatory, antithrombogenic, antidiabetic, anticancer, and neuroprotective activities.

“Sulforaphane” refers to 1-isothiocyanato-4-methylsulfinylbutane. It is produced when the enzyme myrosinase transforms glucoraphanin, a glucosinolate, into sulforaphane upon damage to the plant (such as from chewing), which allows the two compounds to mix and react. Young sprouts of broccoli are particularly rich in glucoraphanin. Sulforaphane has been shown to have anticarcinogenic, antimicrobial and antidiabetic activity.

In some embodiments, a concentrated broccoli extract or nutraceutical composition herein (e.g., a nutraceutical composition comprising a concentrated broccoli extract or a nutraceutical broccoli product comprising edible portions of a broccoli plant) is treated with an S9 liver microsome fraction. An “S9 liver microsome fraction” or “S9 fraction” is the product of an organ tissue. It is defined by the U.S. National Library of Medicine's IUPAC Glossary of Terms Used in Toxicology as the “Supernatant fraction obtained from an organ (usually liver) homogenate by centrifuging at 9000 g for 20 minutes in a suitable medium; this fraction contains cytosol and microsomes.” S9 fractions typically include metabolic enzymes such as aldehyde oxidase, cytochrome P450, flavin monooxygenases, glutathione transferase, monamine oxidase, sulfurotransferase, and uridine glucuronide transferase. Suitable liver S9 fraction may be prepared by conventional methods or obtained from commercial sources such as XenoTech and ThermoFisher Scientific.

Nutraceutical products are also provided herein. As used herein, the term “nutraceutical” refers to a compound, extract, or product that has been isolated, purified, or obtained from food, plants, or edible matter, and that provides a detectable physiological benefit. A nutraceutical product may be a fortified food or a dietary supplement.

In some embodiments, the nutraceutical product comprises a broccoli extract, e.g., a broccoli extract prepared by subjecting a broccoli plant to at least one abiotic stimulus; (b) extracting aerial material of the broccoli plant with a solvent; (c) drying the extract; (d) reconstituting the extract to form a concentrated broccoli extract, the broccoli extract being at a concentration of about 150 mg/ml; and (e) optionally treating the concentrated broccoli extract with an S9 liver microsome fraction.

In some embodiments, the nutraceutical product is a nutraceutical broccoli product comprising an enhanced level of anti-inflammatory compounds, the broccoli product prepared by a method comprising subjecting a broccoli plant to at least one abiotic stimulus, e.g., as described herein, to enhance levels of anti-inflammatory compounds (e.g., sinapates and/or flavonoids) in the broccoli plant and harvesting edible portions of the broccoli plant, e.g., stem, cotyledons, hypocotyls, inflorescence, leaves, or combination thereof. In some embodiments, the nutraceutical broccoli product comprises fresh broccoli plant material. In some embodiments, the nutraceutical broccoli product comprises dried broccoli plant material. In some embodiments, the nutraceutical broccoli product comprises dried and ground or pulverized broccoli plant material.

The broccoli extract, nutraceutical product comprising a broccoli extract, and/or nutraceutical broccoli product comprising an enhanced level of anti-inflammatory compounds may be encapsulated in exosomes. The term “exosome” refers to cell-derived vesicles having a diameter of between about 20 nm to 140 nm, such as between 40 nm and 120 nm, preferably a diameter of about 50 nm to 100 nm, for example, a diameter of about 60 nm, 70 mu, 80 nm, 90 nm, or 100 mm. Exosomes may be isolated from any suitable biological sample from a mammal, including but not limited to, whole blood, serum, plasma, urine, saliva, breast milk, cerebrospinal fluid, amniotic fluid, ascitic fluid, bone marrow and cultured mammalian cells (e.g. immature dendritic cells (wild-type or immortalized), induced and non-induced pluripotent stem cells, fibroblasts, platelets, immune cells, reticulocytes, tumor cells, mesenchymal stem cells, satellite cells, hematopoietic stem cells, pancreatic stem cells, white and beige pre-adipocytes and the like). Exosomes of use herein may be prepared as described herein or by any suitable conventional method, see, e.g., Sun, et al. (2010) Mol. Ther. 18:1606-1614.

As one of skill in the art will appreciate, cultured cell samples may be in the cell-appropriate culture media (using exosome-free serum). Exosomes include specific surface markers absent in other vesicles, including surface markers such as tetraspanins, e.g., CD9, CD37, CD44, CD53, CD63, CD81, CD82 and CD151; targeting or adhesion markers such as integrins, ICAM-1, EpCAM and CD31; membrane fusion markers such as annexins, TSG101, ALIX; and other exosome transmembrane proteins such as Rab5b, HLA-G, HSP70, LAMP2 (lysosome-associated membrane protein) and LIMP (lysosomal integral membrane protein).

Exosomes may also be obtained from a non-mammal or from cultured non-mammalian cells. As the molecular machinery involved in exosome biogenesis is believed to be evolutionarily conserved, exosomes from non-mammalian sources include surface markers which are isoforms of mammalian surface markers, such as isoforms of CD9 and CD63, which distinguish them from other cellular vesicles. As used herein, the term “mammal” is meant to encompass, without limitation, humans, domestic animals such as dogs, cats, horses, cattle, swine, sheep, goats and the like, as well as non-domesticated animals such as, but not limited to, mice, rats and rabbits. The term “non-mammal” is meant to encompass, for example, exosomes from microorganisms such as bacteria, flies, worms, plants, fruit/vegetables (e.g., corn, pomegranate) and yeast.

Extracts and nutraceuticals (i.e., cargo) may be encapsulated within exosomes as described herein and/or by any other suitable method, e.g., sonicating the extract or nutraceutical with an exosome preparation and incubating the formulation in saline at room temperature for about an hour, and optionally separating the exosomes from unencapsulated extract or nutraceutical and debris, e.g., by sucrose gradient separation. Cargo may be also introduced into exosomes, for example, using electroporation applying voltages in the range of about 20-1000 V/cm. Introduction using cationic lipid-based transfection reagents may also be used to introduce cargo into exosomes. Examples of suitable reagents include, but are not limited to, Lipofectamine® MessengerMAX™ Transfection Reagent, Lipofectamine® RNAiMAX Transfection Reagent, Lipofectamine® 3000 Transfection Reagent, or Lipofectamine® LTX Reagent with PLUS™ Reagent.

As will be appreciated by one of skill in the art, prior or subsequent to loading with cargo, the exosomes may be further altered by inclusion of a targeting moiety to enhance the utility thereof as a vehicle for delivery of cargo. In this regard, exosomes may be engineered to incorporate an entity that specifically targets a particular cell to tissue type. This target-specific entity, e.g., peptide having affinity for a receptor or ligand on the target cell or tissue, may be integrated within the exosomal membrane, for example, by fusion to an exosomal membrane marker using methods well-established in the art.

In addition to exosomes, the extracts and nutraceuticals may be loaded into other particle carriers, e.g., nanoparticles, microparticles, and the like. In addition, the exosomes or other particle carriers may be loaded with the extracts and nutraceuticals herein either alone or in combination with one or more other anti-inflammatory compounds.

Broccoli extracts and broccoli-based nutraceuticals (e.g., a nutraceutical product comprising a broccoli extract/encapsulated broccoli extract or a nutraceutical broccoli product comprising an enhanced level of anti-inflammatory compounds) find particular use in treating inflammation, treating cancer, and/or preventing cancer recurrence in a subject.

Accordingly, in some embodiments, a method of treating inflammation in a subject in need thereof is provided, which comprises administering to the subject an effective amount of a broccoli extract and/or broccoli-based nutraceutical to the subject thereby treating the subject's inflammation. In some embodiments, the inflammation is associated with an inflammatory disease or condition. In an embodiment, the inflammatory disease or condition is rheumatoid arthritis, juvenile chronic arthritis, osteoarthritis, ankylosing spondylitis, psoriasis, psoriatic arthropathy, inflammatory bowel disease, psoriatic arthropathy, Reiter's syndrome, Adult Still's disease, Behcet's syndrome, Crohn's disease, multiple sclerosis, ulcerative colitis, uveitis, inflammatory lung disease, muscular dystrophy, lupus, allergy, asthma or chronic obstructive pulmonary disease.

In other embodiments, a method of treating or reducing the risk of cancer recurrence in a subject is provided, which comprises administering to the subject an effective amount of a broccoli extract and/or broccoli-based nutraceutical to the subject thereby treating or reducing the risk of cancer recurrence in the subject. In some embodiments, the cancer is prostate cancer, liver cancer, colon cancer, brain cancer, or bladder cancer.

The term, “subject,” “subjects,” or “subjects in need thereof” include humans as well as non-human subjects, particularly domesticated and farm animals. The terms “treat,” “treating,” or “treatment” as used herein and as well understood in the art, mean an approach for obtaining beneficial or desired results, including without limitation clinical results in a subject being treated. Beneficial or desired results can include, but are not limited to, alleviation or amelioration of one or more signs or symptoms of a condition, diminishment of extent of disease, stabilizing (i.e., not worsening) the state of a disease or condition, delaying or slowing of disease progression, amelioration or palliation of the disease state, diminishment of the reoccurrence of disease, and remission (whether partial or total), whether detectable or undetectable. For example, where the physiological state is inflammation, “treatment” refers to reducing inflammation of a treated subject, e.g., as evidenced by a decrease in expression of inflammatory marker proteins. As another example, where the physiological state is cancer, the term “treatment” refers to reducing tumor mass, slowing the progression of cancer or decreasing the risk of cancer recurrence in a subject. Treatment of inflammation means reducing the inflammatory response either systemically or locally within the body. “Treat,” “treating,” and “treatment” can also mean prolonging survival as compared to expected survival if not receiving treatment and can be prophylactic. Such prophylactic treatment can also be referred to as prevention or prophylaxis of a disease or condition. The prophylaxis may be partial or complete. Partial prophylaxis may result in the delayed onset of a physiological condition. The person skilled in the art will recognize that treatment may, but need not always, include remission or cure.

The term “effective amount” refers to the amount of the active ingredient, the extract or nutraceutical, to be administered to the subject to trigger the desired effect without or causing minimal toxic adverse effect against the subject. One skilled in the art should know that the effective amount can vary from one individual to another due to the external factors such as age. sex, disease state, races, body weight, formulation of the extract, availability of other active ingredients in the formulation and so on.

In some embodiments, the compositions and methods comprise the use of about 1 pg to about 10 g, e.g., about 250 pg to about 5 g, about 500 pg to about 1 g, about 600 pg to about mg, about 750 pg to about 400 mg, or about 1 mg to about 300 mg of broccoli extract or broccoli-based nutraceutical.

Broccoli extracts and broccoli-based nutraceuticals (e.g., a nutraceutical product comprising a broccoli extract/encapsulated broccoli extract or a nutraceutical broccoli product comprising an enhanced level of anti-inflammatory compounds) may be formulated and/or provided in a unit dose form for use in the methods described herein. As used herein, “formulated,” “formulation,” or “composition” may be used interchangeably and refer to a combination of at least two ingredients. In some embodiments, at least one ingredient may be an active agent or otherwise have properties that exert physiologic activity when administered to a subject.

The formulation or composition may be prepared in a number of ways, including as a dry powder, as a capsule, as a ready to drink juice or as a food additive. Alternatively, the formulation may also be brewed, fermented, boiled to create a compote, prepared as an alcohol or water extract. The formulation may be packaged and sold as a powder. The powder may then be reconstituted with water or blended with foods and thereby be ingested for medicinal, preventive, nutritional, or otherwise health-related purposes. Alternatively, the powder may be encapsulated. The capsule may then be swallowed or broken open to be used as the powder described above. Further, the powder may be formed into a solid pill. The solid pill may be dissolved in water for intake, crushed into a powder as described above, or it can be swallowed.

The broccoli extracts and broccoli-based nutraceuticals may be incorporated into solid compositions. Solid compositions may include conventional nontoxic solid carriers including, for example, pharmaceutical grades of mannitol, lactose, starch, magnesium stearate, sodium saccharin, talc, cellulose, dextran, glucose, sucrose, magnesium carbonate, and the like.

Liquid compositions can, for example, be prepared by dissolving, dispersing, and the like, a broccoli extract or broccoli-based nutraceutical as described herein and optional adjuvants in an excipient, such as, for example, water, saline, aqueous dextrose, glycerol, ethanol, and the like, to thereby form a solution or suspension. If desired, the liquid composition may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art; for example, see Remington's Pharmaceutical Sciences, latest edition.

In some embodiments, the broccoli extracts or broccoli-based nutraceuticals are provided in compositions containing permeation enhancer excipients including polymers such as polycations (chitosan and its quaternary ammonium derivatives, poly-L-arginine, aminated gelatin); polyanions (N-carboxymethyl chitosan, poly-acrylic acid); and, thiolated polymers (carboxymethyl cellulose-cysteine, polycarbophil-cysteine, chitosan-thiobutylamidine, chitosan-thioglycolic acid, chitosan-glutathione conjugates).

For oral administration, the composition may generally take the form of a tablet, capsule, a softgel capsule or maybe an aqueous or nonaqueous solution, suspension, or syrup. Tablets and capsules for oral use may include one or more commonly used carriers such as lactose and corn starch. Lubricating agents, such as magnesium stearate, are also typically added. Typically, the compositions of the disclosure may be combined with an oral, non-toxic, pharmaceutically acceptable, inert carrier such as lactose, starch, sucrose, glucose, methyl cellulose, hypromellose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, dextran, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Formulations for buccal administration include tablets, lozenges, gels and the like. Alternatively, buccal administration can be effected using a transmucosal delivery system as known to those skilled in the art. The broccoli extracts and broccoli-based nutraceuticals of the disclosure may also be delivered through the skin or mucosal tissue using conventional transdermal drug delivery systems, i.e., transdermal “patches” wherein the agent is typically contained within a laminated structure that serves as a drug delivery device to be affixed to the body surface. In such a structure, the drug composition is typically contained in a layer, or “reservoir,” underlying an upper backing layer. The laminated device can contain a single reservoir, or it can contain multiple reservoirs. In one aspect, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, can be either a polymeric matrix as described above, or it can be a liquid or gel reservoir, or can take some other form. The backing layer in these laminates, which serves as the upper surface of the device, functions as the primary structural element of the laminated structure and provides the device with much of its flexibility. The material selected for the backing layer should be substantially impermeable to the active agent and any other materials that are present.

When liquid suspensions are used, the broccoli extract or broccoli-based nutraceutical may be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, are not limited to, preservatives, suspending agents, thickening agents, and the like.

The broccoli extract or broccoli-based nutraceutical may be formulated as an ointment or cream. Ointments are semisolid preparations which are typically based on petrolatum or other petroleum derivatives. Creams containing the selected active agent, are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating, and nonsensitizing.

In some embodiments, the broccoli extracts or broccoli-based nutraceuticals are provided in a food composition or food additive. The food composition may be used for ameliorating or preventing inflammation or cancer or cancer recurrence due to its antioxidant activity. In this case, the broccoli extract (e.g., including sulforaphane) may be added without further processing or may be used together with other foods or food ingredients in accordance with methods known in the art. The amount of the active ingredient may be suitably determined according to its intended purpose, such as prophylactic, health care or therapeutic purpose. The food composition may further contain one or more additives selected from nutrients, vitamins, electrolytes, flavoring agents, coloring agents, pectic acid and its salts, alginic acid and its salts, organic acids, protective colloidal thickeners, pH adjusting agents, stabilizers, preservatives, glycerin, alcohols, and carbonating agents for carbonated drinks. The food composition may further contain flesh or vegetative pulp for the production of natural fruit juices, fruit juice beverages, and vegetable beverages. Such ingredients may be used independently or as a mixture thereof. The proportions of such additives are not limited but are typically selected from the range of 0.01 to 0.1 parts by weight, based on 100 parts by weight of the composition.

When it is intended to produce a food or beverage, the broccoli extract or broccoli-based nutraceutical is typically added in an amount of 15 parts by weight or less, preferably 10 parts by weight or less, based on 100 parts by weight of the raw materials of the food or beverage. In the case where the food or beverage is taken for a long time for the purpose of health and hygiene or health care, the amount of the broccoli extract or broccoli-based nutraceutical may be adjusted to less than the lower limit defined above. The food composition is free from problems associated with safety because it uses the natural product or broccoli plant extracts consisting solely of naturally-occurring chemicals. Accordingly, the broccoli extract or broccoli-based nutraceutical may also be used in an amount exceeding the upper limit defined above.

There is no particular restriction on the kind of the food. Examples of foods that may be added with the broccoli extract or broccoli-based nutraceutical include all common foods, such as meats, sausages, breads, chocolates, candies, snacks, cookies, pizzas, instant noodles, other noodles, chewing gums, dairy products including ice creams, soups, beverages, teas, drinks, alcoholic beverages, and vitamin complexes.

Beverages may contain one or more additional ingredients, such as flavoring agents or natural carbohydrates, like general beverages. The natural carbohydrates may be monosaccharides, such as glucose and fructose, disaccharides, such as maltose and sucrose, polysaccharides, such as dextrin and cyclodextrin, and sugar alcohols, such as xylitol, sorbitol, and erythritol. The beverage may contain one or more sweetening agents. As the sweetening agents, there may be used, for example, natural sweetening agents, such as thaumatin and stevia extract, and synthetic sweetening agents, saccharine and aspartame.

Extracts and nutraceuticals herein may be formulated for therapeutic use by combination with a pharmaceutically or physiologically acceptable carrier. The expressions “pharmaceutically acceptable” or “physiologically acceptable” means acceptable for use in the pharmaceutical and veterinary arts, i.e., not being unacceptably toxic or otherwise unsuitable for physiological use. As one of skill in the art will appreciate, the selected carrier will vary with intended utility of the formulation. In one embodiment, extracts and/or nutraceuticals are formulated for administration by infusion or injection, e.g., subcutaneously, intraperitoneally, intramuscularly or intravenously, and thus, are formulated as a suspension in a medical-grade, physiologically acceptable carrier, such as an aqueous solution in sterile and pyrogen-free form, optionally, buffered or made isotonic. The carrier may be distilled water (DNase- and RNase-free), a sterile carbohydrate-containing solution (e.g., sucrose or dextrose) or a sterile saline solution comprising sodium chloride and optionally buffered. Suitable saline solutions may include varying concentrations of sodium chloride, for example, normal saline (0.9%), half-normal saline (0.45%), quarter-normal saline (0.22%), and solutions comprising greater amounts of sodium chloride (e.g., 3%-7%, or greater). Saline solutions may optionally include additional components, e.g. carbohydrates such as dextrose and the like. Examples of saline solutions including additional components, include Ringer's solution, e.g., lactated or acetated Ringer's solution, phosphate buffered saline (PBS), TRIS (hydroxymethyl) aminomethane hydroxymethyl) aminomethane)-buffered saline (TBS), Hank's balanced salt solution (HBSS), Earle's balanced solution (EBSS), standard saline citrate (SSC), HEPES-buffered saline (HBS) and Gey's balanced salt solution (GBSS).

In other embodiments, extracts and nutraceuticals are formulated for administration by routes including, but not limited to, oral, intranasal, enteral, topical, sublingual, intra-arterial, intramedullary, intrathecal, inhalation, ocular, transdermal, vaginal or rectal routes, and will include appropriate carriers in each case. For example, extract and/or nutraceutical compositions for topical application may be prepared including appropriate carriers. Creams, lotions and ointments may be prepared for topical application using an appropriate base such as a triglyceride base. Such creams, lotions and ointments may also contain a surface-active agent. Aerosol formulations may also be prepared in which suitable propellant adjuvants are used. Other adjuvants may also be added to the composition regardless of how it is to be administered, for example, anti-microbial agents, anti-oxidants and other preservatives may be added to the composition to prevent microbial growth and/or degradation over prolonged storage periods.

Alternatively, the extract and/or nutraceutical pellet may be stored for later use, for example, in cold storage at 4° C., in frozen form or in lyophilized form, prepared using well-established protocols. The pellet may be stored in any physiological acceptable carrier, optionally including cryogenic stability and/or vitrification agents (e.g., DMSO, glycerol, trehalose, polyhydroxylated alcohols (e.g., methoxylated glycerol, propylene glycol), M22 and the like).

The foregoing may be better understood by the following examples which are presented for purposes of illustration and are not intended to limit the scope of the invention.

### Example 1: Materials and Methods

Chemicals. All chemicals used for plant growth and preparation of extract were obtained from Sigma-Aldrich (St. Louis, MO), and ThermoFisher Scientific (Waltham, MA).

Plant Material. Planting and preparation of plant material were performed as previously described (Ghura et al. (2016) Sci Rep. 6:29364). Briefly, broccoli seeds (Brassica oleracea var. italica, cv obtained from Eden brothers (Arden, NC) and Johnny's Selected Seeds (Winslow, ME)) were sown (500 seeds/tray) and grown in complete darkness on 0.5× Murashige and Skoog media (no sucrose) 0.8% agarose phytatrays, then sealed in black acrylic (Plexiglas®) boxes stored in 4° C. for 48 hours. Cold-vernalized phytatrays were then moved to continuous darkness at 20° C. for 5 days (Orozco-Nunnelly et al. (2014) PLOS ONE 9:e93371). Plant growth and treatment procedures were performed as previously described (Ghura et al. (2016) Sci Rep. 6:29364), with the following modifications. Broccoli seedlings were treated with sublethal UV treatments of 254 nm, and 315 nm (Warpeha et al. (2008) Plant Cell Environ. 31:1756-70), each at a fluence (dose) of 10⁴ μmol m⁻², followed by cold (1 hour at 4° C.) and blue light (10⁴ μmol m⁻²), with 1 minute between treatments on day 5, then phytatrays returned to darkness for 12 hours. In the first instance, the primary leaves were harvested live and subjected to maceration in 90% ethanol, then allowed to sit 1 hour at 4° C. Debris was pelleted and supernatant read by spectrophotometer (SmartSpec™ Bio-Rad, Hercules, CA). Heirloom cultivars were checked for content of sinapates and flavonoids based on conditions for extraction (Shi et al. (2019) Spine J. 19:171-81) (Table 1).

TABLE 1

| Heirloom Broccoli type | Sinapic esters* | Polyphenols |
|---|---|---|
| Calabrese | .32 | .21 |
| De Cicco | .24 | .11 |
| Purple Sprouting | .22 | .20 |
| Rudolph | .19 | .14 |
| Waltham | .18 | .13 |
| Romanesco | .18 | .13 |

Estimated phenylpropanoid peak area is proportional to metabolite quantity and is just a first screen after UV (317 nm, 254 nm) and cold induction (1 hour at 4° C.), estimated on BioRad SmartSpec ™ Spectrophotometer, confirmed by fluorescence.
*Peak association with metabolites based on wavelength and ethanol extraction (Shi et al. (2019) Spine J. 19: 171-81). Extraction solubles based on plant materials and conditions described herein.

The heirloom broccoli Calabrese became the focus for this study. Calabrese seeds were sown and grown and, at day 5, treated as described above with abiotic stimuli. Seedling leaf tissue was harvested 12 hours after the last abiotic treatment and placed in liquid nitrogen. The frozen leaf material was ground to a powder and stored at −80° C. Samples were maintained at −80° C. until extracted in 90%-95% ethanol (referred to as OE). In some instances, OE was further processed by treatment with $9 liver fraction (referred to as S9-treated OE) to mimic liver processing.

Plant Material Extraction. Extraction was performed with ethanol (Orozco-Nunnelly et al. (2014) PLOS ONE 9:e93371) with some modifications. In particular, the aerial portions of Calabrese were ground in liquid nitrogen, weighed, and extracted twice for 2 hours with 95% aqueous ethanol by sonication (20 mL of solvent per gram of plant material). After filtration, the filtrate was evaporated to remove the solvent using a rotary evaporator under a reduced vacuum, then freeze-dried. Freeze dried material (150 mg/mL) was resuspended in DMSO 1:2 for storage, and later dimethylsulfoxide (DMSO) samples were further diluted (referred to as OE) in phosphate-buffered saline (PBS) for experiments, with 1:200 dilution used for mouse behavior studies being 375 μg/mL.

Rabbit Disc Cell Isolation, Culture and Treatment. Rabbit disc tissues were isolated from New Zealand white rabbit spines, obtained from the animal tissue program at Rush where investigators donate unused tissues from one study to others to help conserve the number of animals used in research. Cells were isolated by sequential enzymatic digestion, seeded in tissue culture plates and grown in monolayers in complete media (DMEM/F12 (Mediatech, Manassas, with 50 μg/mL gentamicin (Gibco, VA) supplemented ThermoFisher Scientific), 25 μg/mL ascorbic acid (Sigma-Aldrich, St. Louis, MO) 20% fetal calf serum (Omega Scientific, Tarzana, CA)) in a 5% CO₂ incubator at 37° C. After cells reached 80% confluency, they were cultured for 24 hours in starvation media (DMEM/F12 supplemented with 1% insulin, human transferrin, and selenous acid (ITS, Corning Life Sciences, Tewksbury, MA), L-glutamine, gentamicin and ascorbic acid), and then treated with lipopolysaccharide (LPS; 100 ng/ml; Sigma) in the presence or absence of different concentrations of broccoli OE for 16 hour. Control treatments were composed of starvation media alone and were included in all experiments. Conditioned media were collected for protein analysis and cell pellets for RNA analysis.

Conditioned media analysis of rabbit Interleukin-8 (IL-8). Conditioned media were collected and stored at −80° C. until analysis. Rabbit IL-8 levels were measured using enzyme-linked immunosorbent assay kit for rabbit IL-8 (Raybiotech, Norcross, GA) following the manufacturer's instructions.

Surgical Methods for Rabbit Disc Degeneration and Treatment. New Zealand white rabbits (Charles River Laboratories, Wilmington, MA) weighing about 2.8-3.0 kg were used in this study (n=8 total). The rabbits were cared for and maintained in accordance with National Institute of Health guidelines. All studies were approved by Rush University Medical Center's Institutional Animal Care and Use Committee. Under general anesthesia and using aseptic conditions, a left abdominal incision was made and the ventral surface of four consecutive lumbar IVDs (L3/4, L4/5, and L5/6) was exposed. Using an 18-gauge needle with a stopper device that allows the needle to go to a depth of 5 mm, the annulus fibrosus was punctured in the ventral aspect into the nucleus pulposus (NP) at all three levels. Suction was applied for the removal of NP tissues. Rabbits were randomized and discs were treated during the same operation of puncture. Eight microliters (8 μL) of OE (n=4) or saline (0.9% sodium chloride, Baxter Healthcare Corporation, Deerfield, IL; n=4) were injected into the center of the injured discs with a 26-gauge needle. The surgical wound was repaired in layers. Meloxicam (1.5 mg) was also given orally (one day before surgery and 2-3 days after the operation). An analgesic (buprenorphine HCl 0.01-0.03 mg/kg) was given up to twice daily for 2-3 days when needed in consultation with the veterinary staff. After recovery from anesthesia, the rabbits were returned to their cages where they mobilized ad lib. At 3 weeks post-surgery and treatment, animals underwent euthanasia, and discs were isolated for RNA analysis.

Isolation of Total RNA and Measurement of mRNA Levels with Real-Time Polymerase Chain Reaction (Real-Time PCR). Total RNA was processed from cell pellets and intervertebral disc tissues. RNA isolation and real-time PCR analysis was performed as described (Zhang et al. (2015) Am. J. Phys. Med. Rehabil. 94:530-8). Taqman® gene expression assays were used for analysis or normalization of the following genes: rabbit collagen type I (COL1), collagen type II (COL2), IL-8, hypoxanthine phosphoribosyltransferase 1 (HPRT1), and 18S rRNA (Oc03396113_ml, Oc03396134_ml, Oc03397860_ml, Oc03399461_ml, Hs99999901_s1, respectively; all from Applied Biosystems, Foster City, CA). PrimeTime® Mini qPCR Assays were designed for rabbit nitric oxide synthase 2 (NOS2), chemokine C-C motif ligand (CCL) 2, and CCL5 (Integrated DNA Technologies, Coralville, IA) as described (Chou et al. (2020) Spine J. 20:2025-36). DataAssist™ Software was used to calculate the relative gene expression using the comparative CT (ΔΔCT) method.

Treatment of OE Using S9 Liver Microsome Fraction. S9 fraction (S9) is a sub-cellular fraction of liver microsomes that contains both phase 1 and 2 metabolizing enzymes (Richardson et al. (2016) Drug Metab. Lett. 10:83-90). To mimic liver processing and to improve bioavailability (Richardson et al. (2016) Drug Metab. Lett. 10:83-90) the OE was treated with mouse liver S9 (Gibco, ThermoFisher Scientific) according to the manufacturer's instructions with some modifications, using the protocol at four times the recipe (volumes). Solubilized OE at 1:2 dilution (75 mg/mL) was added as the ‘test article’ volume×2 so the total of OE was 4 μL in the assay (total single assay volume is 200 μL). After the supernatant was removed, it was spun one additional time and the supernatant, which was 100 μL, was saved and composed of approximately a 50-fold dilution of the OE. This treated extract was labeled the S9-treated OE, and was stored in −20° C. When used in a mouse behavior study, the S9-treated OE was diluted in PBS to be equivalent to 1:200 dilution (375 μg/mL) of extract, with i.p. of 20 μL. No organic solvent was used in the preparation of S9-treated OE. The S9-treated OE was used in experiments with live mice in vivo at room temperature.

Mouse Activity Studies. Six-month-old C57BL/6J adult male (n=4) and female (n=4) mice (Charles River Laboratories, Wilmington, MA) were used for general activity monitoring, and pain behavior tests as a result of formalin. Animals were kept in a temperature-controlled environment of 22° C. with a 12-hour light/dark cycle (Browe et al. (2020) Neurobiol. Pain 8:100047). All procedures were conducted according to the animal protocols approved by the University of Illinois at Chicago Institutional Animal Care and Use Committee. To control for the possibility that the extract might reduce activity in general and not specifically pain behaviors, the open-field paradigm was used as a measure of activity (Gellert & Varga (2016) Bio. Protoc. 6:e1857). Mice were placed one at a time into an opaque plexiglass arena and allowed to freely explore. The arena floor was demarcated into 36 (6×6) equal square regions of 5×5 inches, or 12.5 cm each. The number of times a mouse completely crossed from one region to another was recorded over a 10-minute period. On day 1, half of the mice were given OE (20 μL) via i.p. injection. The other half of the mice were given an equal volume (20 μL) of vehicle control (VC). Testing commenced 30 minutes later. On day 2, the mice that had been given OE received vehicle, and the mice that had been given vehicle received OE. A paired t-test was used to compare the number of regional crossings for extract treatment versus vehicle treatment. There was no difference in activity for VC and OE. Hence, data in response to pain stimulus is not a result of activity changes.

Mouse Pain Tests. All mice were randomized, then received either intraperitoneal (i.p.) 20 μL (1:200; 375 μg/mL) extract or vehicle control (VC) (1:200 DMSO in sterile PBS) 30 minutes prior to subcutaneous injection of 20 μL (2%) formalin to the hind paw using a 30-gauge insulin syringe. The formalin was test performed as described with modifications (Browe eta 1. (2020) Neurobiol. Pain 8:100047; Eigenbrod et al. (1979) Science 2019 (364): 852-9). Upon injection, the timer started for behavioral observations. Animals were placed into a standard empty mouse cage and observed for 70 minutes. Licking of the formalin-injected foot was operationally defined as pain behavior. The total time was divided into 0-5 minute (Phase I) defined as early phase, and 40-55 minutes (Phase II) defined as late phase. The test was performed and scored by observers blinded to experimental conditions. Pain behavior tests were performed on two different days with a 4-day gap between VC or extracts. Animals were randomized into treatment (OE or S9-treated OE) or control groups and switched for testing after the 4-day gap.

Statistical Analyses. All statistical analyses were carried out using GraphPad Prism v. 9 (GraphPad Software, San Diego, CA), with suggested tests from GraphPad Prism after determination that data followed a normal distribution. For all tests, differences were considered significant when the P value was less than 0.05 (P<0.05). Rabbit Experiments. Gene expression and protein analysis were analyzed between treatment groups with One-way ANOVA, with multiple comparisons. In vivo rabbit disc gene expression was analyzed with multiple unpaired t-tests. In vivo Mouse Data. All data in this paper are represented as mean±SEM. Unpaired t-test with Welch's correction was performed for the groups in the mouse behavior tests (early and late pain behavior), where vehicle control was compared to treatment (OE or S9-treated OE) for early and late responses as shown on the figures. *P<0.05, **P<0.01, ***P<0.001. Exosome Studies. Data obtained are presented as the mean with standard deviation. Statistical analyses were carried out using OriginLab software, GraphPad Prism, and Microsoft Excel. Using a one-way analysis of variance, the significance of the evaluated groups was examined (One-way-ANOVA). Post test analysis used are mentioned in the appropriate results subsections.

HPLC and Mass Spectrometry (MS). Pre- and post-treatment extracts were analyzed using HPLC-UV-MS/MS to identify the chemical constituents. The LC-MS method was developed based on the previously described analysis (Ghura et al. (2016) Sci Rep. 6:29364). Sample preparation involved removal of solvents from pre- and post-treatment extracts (DMSO and PBS, respectively) by water wash on a C18 cartridge, followed by sample elution with methanol. LC-MS samples were prepared in LC-MS-grade methanol and filtered through 0.2 μm membrane filters. A Shimadzu UFLC system coupled with an LCMS-2020 detector, and an Agilent EC-C18 column (3.0×150 mm, 2.7 μm) protected with a pre-column (3.0×5 mm), were used for the HPLC-UV-MS analyses. Formic acid (0.1%) in water (A) and 0.1% formic acid in acetonitrile (B) were used as mobile phase and run under the gradient program as follows: 5% B (0-1 minute), 5-15% B (1-2 min), 15-30% B (2-29 minutes), 30-100% B (29-30 minutes), 100% B (30-35 minutes). Flow rate was maintained at 0.4 mL/minute and column oven temperature was set at 27° C. The LC-MS/MS analyses were performed on a Bruker Impact II quadrupole/time-of-flight (Q/TOF) mass spectrometer coupled with a Shimadzu UHPLC system under similar chromatographic conditions. Bar charts were created using Origin 2020, Version 9.7 (OriginLab Corporation, Northampton, MA) software.

Cell Lines and Maintenance. Murine macrophages (RAW 264.7 cells), purchased from ATCC were used for the in vitro analysis of exosome formulations. The cells were cultured and maintained in a 25 cm² tissue culture flask containing 10% FBS in DMEM with 1% penicillin-streptomycin (Pen-Strep) with a seeding density of 1×10⁶ cells/mL. When RAW 264.7 cells reached 80-90% confluency, the adherent cells were sub-cultured using a cell scraper to gently remove cells from the flask. The cell suspension was then centrifuged at 1200 rpm for 5 minutes to obtain a pellet, which was w further resuspended in 1 mL of fresh medium. The cells were maintained in an incubator at 37° C. with 5% CO₂. The growth medium was changed on alternate days.

Exosome Isolation. RAW 264.7 cells were grown in 20% DMEM medium until they reached 70% confluency. The medium was subsequently replaced, and the cells were incubated with exosome-free FBS containing 10% DMEM for 24 hours. The cell culture medium was harvested the next day and centrifuged at 2000×g for 30 minutes to remove cell debris. The cell medium was mixed with a total Exosome Isolation Reagent (Invitrogen) and incubated at 4° C. overnight. The next day, the mixture was centrifuged at 10,000×g for 60 minutes to isolate the precipitated exosomes. The exosomes were then prepared for downstream analysis by resuspending the pellet in PBS or a comparable buffer.

Dynamic Light Scattering (DLS). The size distribution profile of the exosomes was characterized using dynamic light scatter (DLS Malvern Instruments Nano ZS90; Westborough, MA). The exosomes were resuspended in polystyrene cuvettes after being diluted in deionized water. The mean particle size for the sample was subsequently calculated.

Exosome Quantification. Exosomes were precipitated and lysed in 20-30 μl (depending upon the pellet) of lysis buffer provided in the EXOCET exosome quantification kit (System Biosciences). Lysed exosomes were incubated at 37° C. for 5 minutes to liberate exosomal proteins. The sample was then sonicated for 20 seconds and kept on ice for 20 seconds, and this step was repeated 3 times. The last incubation on ice was performed for 15 minutes and the sample was centrifuged at 1500×g for 5 minutes to remove all the debris. The supernatant was transferred to a fresh microcentrifuge tube. To quantify the protein, 1 μL of the sample was diluted using 49 μL of PBS. To each clear well of a 96-well plate was added: 50 μL of Reaction buffer (provided in the kit) and 50 μL of Standard or Exosome sample to make up a total of 100 μL reaction volume. The plate was incubated at room temperature for 20 minutes and the plate was read using the spectrophotometric plate reader immediately at 405 nm. A standard curve was plotted and the concentration of exosomes in the sample was calculated.

Western Blot Analysis. Exosomes derived from RAW 264.7 cells were lysed using RIPA buffer followed by protein quantification using the bicinchoninic acid assay (BCA assay). A total of 40 μg of sample proteins was mixed with an equal volume of Laemmli buffer (Bio-Rad Laboratories, Hercules, CA). The sample was boiled for 5 minutes, and the protein was separated via a 12% sodium dodecyl sulfate-polyacrylamide gel. The separated proteins were transferred onto a PVDF membrane which was blocked with 5% skim milk overnight in the cold room. After blocking, the blot was washed three times with phosphate-buffered saline Tween® buffer (PBST) and incubated overnight with primary antibodies, i.e., anti-mouse β-actin (Cell Signaling Technology) diluted 1:1000 (control protein), rabbit anti-mouse mAb CD9 (Cell Signaling Technology) at a dilution of 1:1000, and rabbit anti-mouse mAb HSPA8 (Cell Signaling Technology) at a dilution of 1:1000. The blot was washed in PBST and incubated at 4° C. for 5 hours with the secondary antibodies, i.e., Goat Anti-Rabbit IgG-HRP antibody (Cell Signaling Technology) at a dilution of 1:2000 and Goat Anti-Mouse IgG-HRP (Cell Signaling Technology) diluted 1:2000. The blots were washed with PBST, and bands were detected by FEMTO Western Blotting System (Thermofisher). Bands were visualized on a Chemidoc imaging system.

Cryo-Transmission Electron Microscopy (CryoTEM). The imaging was carried out at Northwestern University, IL. Grids were glow-discharged before loading 4 μL of sample. To visualize the exosomes, Cryo-TEM was performed by using JEOL TEM-1230 Electron Microscope (Peabody, MA).

Preparation of Exosome Encapsulated Extract. Different methods were tested to load the broccoli extract into the exosomes. In each of the methods, 5 μL of the OE was added to the isolated exosomes (1×10⁹/μL). The loading efficiency was checked for by UV5 Nano spectroscopy. In the first method, the broccoli extract-loaded exosomal samples were sonicated for 6 cycles of 30 seconds on/off for 2 minutes with a cooling period between each cycle. After all 6 cycles were complete, the formulation referred to as “EXO/PA-S” was incubated at 37° C. for 1 hour. In the second method, the extract was incubated with exosomes at room temperature for 1 hour in a shaking incubator at a minimal speed of 70-75 rpm. This second formulation was referred to as “EXO/PA-I.”

UV Spectroscopy. The loading efficiency of the broccoli extract-loaded exosomes was determined by different methods. A standard curve was obtained by measuring the UV absorbance of broccoli extract over a range of different concentrations. The standard curve graph was used to determine the loading efficiency of the exosomes. Dialysis was performed to remove the unencapsulated broccoli extract from the formulation. The broccoli extract-loaded exosome nanocarriers were lysed using Triton™-X 100 (0.01%) to quantify the amount of broccoli extract in the nanoparticles. Using a UV spectrophotometer to measure the amount of broccoli extract encapsulated, the encapsulation efficiency (EE) of the phenylpropanoids in exosomes was determined by Equation 1.

$$EE \% = 1 - \frac{[ Initial absorbance of 1 : 10 broccoli extract - Final absorbance of broccoli extract in exosomes ]}{[ Initial absorbance of 1 : 10 broccoli extract ]} * 100 \qquad ( Eq . 1 )$$

To assess the stability of the exosomes, the change in size of exosomes as well as the EXO/PA-S and EXO/PA-I were evaluated using DLS. The samples were stored at −80° C. and the size assessment was performed on days 1, 20, and 40. Throughout the course of the study, the samples were kept resuspended in PBS, (pH 7.4)

To analyze the stability of broccoli extract, the broccoli extract-encapsulated exosomes (EXO/PA-S, and EXO/PA-I) were kept at room temperature for over a period of 24 hours and the exosomes were lysed and the absorbance of the released broccoli extract was assessed at 0 hour, 1 hour, 15 hours, and 24 hours using a UV spectrophotometer.

Biocompatibility Assay. For further experiments, cells were seeded into 96-well plates in 100 μL/well of complete media (10% FBS in DMEM and 1% Pen-Strep) and incubated for 24 hours at a seeding density of 1×10⁴ cells/well. After 24 hours, the adhered cells were treated at 1:50 dilution of the broccoli extract-encapsulated exosomes. In the case of non-treated control cells, the media was replaced with fresh medium, and for positive control, the cells were treated with bare broccoli extract (the same dilution that was found encapsulated in the EXO/PA-S and EXO/PA-I nanocarriers). After incubation of 24 hours, the spent media was removed from each well of the 96-well plate containing pre-treated cells and replaced with 100 μL of 10% alamarBlue solution in complete media (comprising 10% FBS in DMEM with 1% penicillin-streptomycin) in each well. The plate was incubated for 4 hours at 37° C. and 5% CO₂, then the plate was observed for a color change, and absorbance of the plate was read at the optical density (OD) of 570 and 600 nm to monitor cell activity.

In Vitro Uptake Analysis. Isolated exosomes were labeled with 2 μg/mL DiL dye for 20 minutes at 37° C. in the dark. After incubation, the exosomes were pelleted at 25, 800 RPM using ultracentrifugation for 2 hours. Then the pellet was washed by PBS using ultracentrifugation again at 25, 800 RPM for 2 hours. The pellet was then resuspended in PBS and run through the Sephadex® column to separate unbound dye and exosomes labeled with DiL dye.

RAW 264.7 macrophages were cultured on coverslips in 12-well plates. 5 μL of DiL labeled exosome with 500 μL DMEM supplemented with 10% exosome-free FBS were incubated with the cells and cultured for 3 hours. The coverslips were then removed with the help of forceps and cells were rinsed with PBS. Cells were stained with Hoechst 33342 nucleic acid stain at a concentration of 1 μg/mL in PBS for 15 minutes. Cells were then fixed for 20 minutes with 3.7% paraformaldehyde. Paraformaldehyde was removed by washing with PBS. The coverslips were mounted onto the slides using a mounting medium and imaged using Keyence confocal microscope.

Apoptosis Assay. RAW 264.7 cells (10⁶) were seeded in a 6-well plate. Right after they reached 70% confluency, the media was replaced with the exosome-free FBS-containing medium. The next day, the cells were treated with the EXO/PA-S and EXO/PA-I nanocarriers for 24 hours. Once ready, the cells were pelleted, and the pellet was washed with PBS. This was followed by mixing 400 μL of the cells+100 μL of incubation buffer+5 μL Annexin V (1 mg/ml)+5 μL of Propidium Iodide (1 mg/mL). The cells were then analyzed using a flow cytometer.

Nitrate Production. The immune-suppressive potential of the broccoli extract-encapsulated exosomes was assessed by analyzing nitric oxide (NO) production, pro-inflammatory gene expression, and cytokine secretion. The Griess reaction, commonly known as the diazotization test, relies on the transformation of nitrite into an azo-dye that is purple in color and can be measured spectrophotometrically at a wavelength of about 540 nm. NO is regarded as a pro-inflammatory mediator that, when overproduced under atypical circumstances, causes inflammation. For the experiment, cells were seeded into 96-well plates in 100 μL/well of complete media (10% FBS in DMEM and 1% Pen-Strep) and incubated for 24 hours at a seeding density of 1×10⁴ cells/well. After 24 hours, the adhered cells were treated with 1:50 dilution of the EXO/PA-S and EXO/PA-I nanocarrier. For control cells, the media was replaced with fresh medium, and in the case of broccoli extract treatment, bare broccoli extract at the same dilution that was found encapsulated in the EXO/PA-S and EXO/PA-I nanocarriers was used. After 24 hours, the cells were then treated with LPS (100 ng/μL) for 24 hours. The next day, 60 μL of the spent media was harvested followed by the addition of 20 μL of the Greiss reagent per well. After a 20-minute incubation at room temperature in the dark, the absorbance of the samples was assessed at 540 nm. A standard curve of NO was used in order to measure the exact amount of NO in the sample.

Gene Expression Analysis. To analyze pro-inflammatory gene expression, RNA was isolated from macrophage cells using an optimized protocol. RAW 264.7 cells (10⁶) were seeded in a 6-well plate. The next day, the cells were treated with the EXO/PA-S and EXO/PA-I nanocarriers for 24 hours. For control cells, the spent media was replaced with fresh media. After 24 hours, the cells were then treated with LPS (100 ng/μL) for 24 hours. After 24 hours, total RNA was isolated, and a high-capacity reverse transcriptase kit was used for the reverse transcription of the obtained mRNA. The quality of the CDNA was evaluated by Glyceraldehyde-3-phosphate dehydrogenase (GAPDH) amplification. The genes selected for inflammatory response evaluation were IL-1B, NFkB, Cox-2, and MMP2. Quantitative real-time reverse transcription-polymerase chain reaction (qRT-PCR) experiments were performed using an Applied Biosystems RT-PCR system (7300-RT-PCR) according to the manufacturer's instructions using the primers listed in Table 2. The levels of expression of each sample were normalized to GAPDH expression and the results were obtained by the comparative method of relative quantification.

TABLE 2

| Primer | | Sequence (5′-3′) | SEQ ID NO: |
|---|---|---|---|
| Mouse-GAPDH | Forward | AGGTCGGTGTGAACGGATTTG | 1 |
| | Reverse | TGTAGACCATGTAGTTGAGGTCA | 2 |
| Mouse-NfkB | Forward | GAGCTGTGCGGTCTGTTAAAG | 3 |
| | Reverse | GGTAGTTCGGTTCCACACCA | 4 |
| Mouse-COX-2 | Forward | ACGGTCCTGAACGCATTTATG | 5 |
| | Reverse | TTGGCCCCATTTAGCAATCTG | 6 |
| Mouse-MMP2 | Forward | CAAGTTCCCCGGCGATGTC | 7 |
| | Reverse | TTCTGGTCAAGGTCACCTGTC | 8 |
| Mouse-IL-1β | Forward | GCACTACAGGCTCCGAGATGAA | 9 |
| | Reverse | GTCGTTGGCTTGGTTCTCCTTGT | 10 |

Cytometric Bead Array (CBA). The release of cytokines (IL-6, TNF-α, and monocyte chemoattractant protein-1 (MCP-1)) by macrophages was measured using the commercial mouse inflammation CBA kit (BD Biosciences). Cells (1×10⁴ cells/well) were seeded in a 96-well plate and allowed to become confluent. The next day, the macrophages were stimulated by adding lipopolysaccharide (LPS) (100 ng/ml) in an exosome-free FBS-containing medium to obtain the cytokine secretion. After 24 hours of LPS stimulation, the cells were treated with different treatment samples, i.e., the broccoli extract-encapsulated exosomes through different methods as mentioned above. CBA was performed after 24 hours of exosomal treatment. Fifty μL of supernatant was incubated with the mouse inflammation capture bead suspension mixture and the PE detection reagent. After 2 hours of incubation, samples were washed and then analyzed using a Becton Dickson (BD) CBA FACS Calibur flow cytometer (BD Biosciences Corp., San Jose, CA). Mouse Inflammation standards provided with the kit, were appropriately diluted and used in parallel with the samples to prepare the standard curves and subsequently calculate the inflammatory response. The analysis of the cytokine secretion was performed using the FCAP Array software.

Evaluation of Mechanistic Pathways. Whole-cell protein was obtained from RAW cells treated with EXO/PA-S and EXO/PA-I nanocarrier for 24 hours. For control cells, the spent media was replaced with fresh media. After 24 hours, the cells were then treated with LPS (100 ng/μL) for 24 hours. The cells were then lysed using RIPA buffer followed by protein quantification using the bicinchoninic acid assay (BCA assay). A total of 20 μg of sample proteins was mixed with an equal volume of Laemmli buffer (Bio-Rad Laboratories, Hercules, CA) and was boiled for 5 minutes. The protein was then separated on the sodium dodecyl sulfate-polyacrylamide gel electrophoresis (SDS-PAGE) gel electrophoresis for which a 128 gel was prepared. Next, the protein was transferred onto a PVDF membrane which was blocked with 5% skim milk for 1 hour. The blot was then washed three times with phosphate-buffered saline tween buffer (PBST) and incubated with the primary antibodies for Vinculin (Cell Signaling Technology) as a control protein, NF-κB (Cell Signaling Technology), IkB, ERK, and p-ERK overnight. The next day the blot was washed in PBST and incubated with the secondary antibodies at room temperature for 1 hour followed by TBST washing. The expected bands were detected by FEMTO Western Blotting System (Thermofisher) and visualized on the Chemidoc imaging system or an X-ray.

### Example 2: Production of a Novel and Stable Broccoli Extract Optimized for Specific Phenylpropanoids

Sprouts have many concentrated nutrients naturally (Gan et al. (2017) Trends Food Sci. Technol. 59:1-14), and broccoli, in particular, has been studied in the case of sulforaphane (Yagishita et al. (2019) Molecules 24:3593; Zhang et al. (1994) Proc. Natl. Acad. Sci. USA 91:3147-50). After testing various abiotic stimuli, a 5-day-sprout protocol was developed that provided for increased phenylpropanoids, as determined by experimental extraction methods. The optimal extraction was an ethanol-based method, where the broccoli sprouts presented a high ratio of flavonoids and sinapates. This original extract, abbreviated herein as “OE,” was resuspended in DMSO, then subsequently diluted in sterile PBS and compared to the vehicle control (VC). Both OE and VC were subsequently tested in rabbit and mouse experimental models of inflammation.

### Example 3: Broccoli Extract Inhibits Inflammation in Rabbit Disc Cells and Induces Disc Regenerative Markers in a Rabbit Model

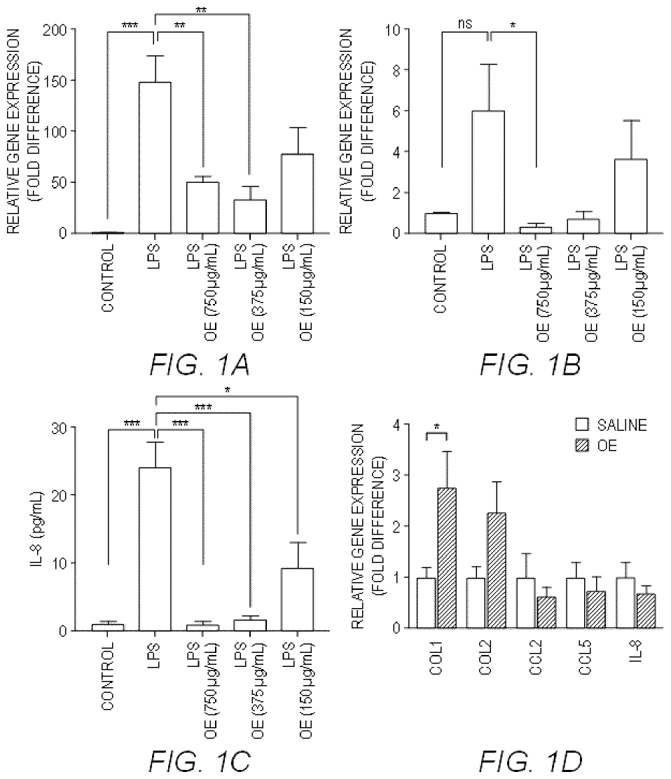

Well-established in vitro studies with disc cells have shown that exposure to lipopolysaccharide (LPS) or inflammatory cytokines can initiate an inflammatory response that leads to the upregulation of catabolic genes associated with disc degeneration (Kedong et al. (2020) Spine J. 20:60-8). It has also been shown that treatment with growth factors can decrease catabolismin disc cells (Takegami et al. (2002) Spine 27:1318-25). To determine the anti-inflammatory effects of OE on disc cells, rabbit disc cells were pre-treated with different concentrations of OE and then exposed to LPS. OE had a dose-dependent effect on reducing gene expression of inflammatory markers interleukin-8 (IL-8; FIG. 1A) and nitric oxide synthase 2 (NOS2; FIG. 1B), and in decreasing the release of IL-8 protein into the conditioned media (FIG. 1C).

A rabbit disc injury model provides an understanding of the biological mechanisms of disc degeneration and may be used to test therapeutics of use in regenerating the disc, e.g., growth factors, cell therapies, and anti-inflammatory molecules. Rabbits (n=8) underwent disc-injury surgery to induce disc degeneration and inflammation and intradiscal treatment with either saline (control) or OE. After 3 weeks, animals were sacrificed, and disc tissues were isolated for gene expression analysis for disc phenotypic and inflammatory markers. OE-treated injured discs showed higher expression of collagen type 1 (COL1) and collagen type 2 (COL2), indicating disc regeneration. Discs treated with OE exhibited decreases in inflammatory gene expression of CCL2, CCL5, and IL-8, but did not achieve significance (FIG. 1D).

### Example 4: Mouse In Vivo Pain Model Indicated that Liver S9-Treated Extract and OE, are Effective at Reducing Pain Behavior

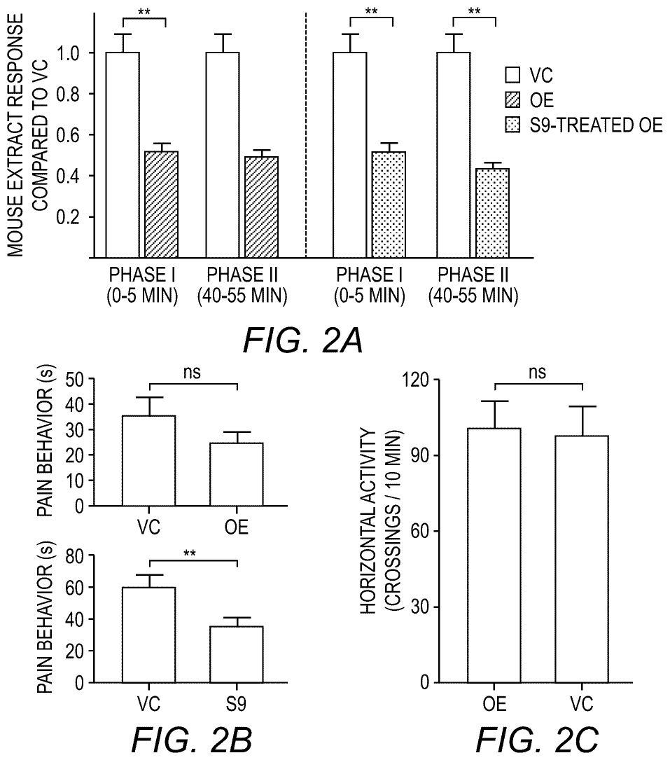

An accepted pain model for mammals' reflexive and non-reflexive inflammation was used to assess pain behavior with formalin as the pain stimulus (Park et al. (2008) PLOS Biol. 6:e13; Browe et al. (2020) Neurobiol Pain 8:100047; Eigenbrod et al. (1979) Science 2019 (364): 852-9; Hunskaar & Hole (1987) Pain 30:103-14). To start the experiment, 30 minutes prior to pain stimulus, a 20 μL i.p. injection of OE or S9-treated OE (375 μg/mL) or vehicle control (VC) was administered to male and female mice. At time zero, the left dorsal hind paw of each mouse was given a subcutaneous injection of formalin (20 μL of 5 mg/mL in 0.1 M PBS). At injection, a dedicated timer was started at time 0 second to record the recognized pain behavior of paw-licking at the site of injection for 70 minutes, counted in brackets of 5 minutes (0-5 minutes, 5-10 minutes, etc.), to reveal early and late behavior (Browe et al. (2020) Neurobiol Pain 8:100047; Eigenbrod et al. (1979) Science 2019 (364): 852-9) (FIG. 2A-2C). OE-treated mice were evaluated for this pain test (4 female, 4 male; total n=8). Pooling the male and female mouse data obtained for OE revealed the early response (Phase I, 0-5 minutes) was significant (P=0.0032), but not the later response (Phase II, 40-55 minutes; P=0.2182) where VC response was set to 1.0 (FIG. 2A). S9-treated OE was evaluated in the same in vivo pain test. Male and female responses were pooled for comparison of S9-treated OE to VC pain response data for early (Phase I, 0-5 minutes; P=0.0018) and late (Phase II, 40-55 minutes; P=0.0062) behavior with VC set to 1.0 (FIG. 2A). Sex differences are possible in pain responses, but when male and female data were separated and compared, no sex differences were observed for either OF early (Phase I, 0-5 minutes; VC P=0.2521, OE P=0.1509) and late (Phase II, 40-55 minutes; VC P=0.1815, OE P=0.8056), or S9-treated OE for early (Phase I, 0-5 minutes; VC P=0.7941, S9 P=0.9848) and late (Phase II, 40-55 minutes; VC P=0.1921, S9 P=0.9828) pain behavior. To better understand the overall responses of pooled male and female mice over the 0-70 minutes testing period, OE and S9-treated OE responses were compared to VC, where S9-treated OE was significant (P=0.0012), but OE was not (P=0.2096) (FIG. 2B). With a different set of mice (4 female, 4 male) of the same age, OE was tested to ensure that there were no alterations of basic activity, where differences between VC and OE were not significant (FIG. 2C). As flavonoids are known for their anti-inflammatory properties, this change could support the observed effects of the S9-treated OE in reducing pain behavior in the mouse pain model, compared to those observed for the OE, even though OE was still effective.

### Example 5: Chemical Constituents in Extracts Indicate Good Candidates for Anti-Inflammatoires and Reveal Processing Information

An HPLC-UV-MS/MS method was adapted for the analysis of the OE extract (Tables 3-4) and S9-treated OE extract (Tables 5-6). Retention time (Rt), UV absorption maxima, MS¹ and MS² data, both in positive and negative mode, were analyzed and compared with literature data (Ghura et al. (2016) Sci Rep. 6:29364; Baumert et al. (2005) Phytochemistry 66:1334-45; Dong et al. (2021) Food Chem. 365:130493; Engels et al. (2012) Eur. Food Res. Technol. 234:535-42; Ferreres et al. (2007) Food Chem. 101:549-58; Siger et al. (2013) Eur. J. Lipid Sci. Technol. 115:1130-8; Wolfram et al. (2010) Phytochemistry 71:1076-84) for tentative identification of the metabolites (Tables 3-6).

TABLE 3

| | | Rt | |
|---|---|---|---|
| Peak | Identification | (min) | UV max (nm) |
| 1 | Kaempferol-sinapoyl- | 7.4 | 222, 268, 331 |
| | tetrahexoside | | |
| 2 | Kaempferol-sinapoyl- | 7.9 | 223, 268, 331 |
| | trihexoside | | |
| 3 | Sinapoyl-hexoside | 8.5 | 235, 331 |
| 4 | Neoglucobrassicin | 11.7 | 223, 268, 299, |
| | | | 320ª |
| 5 | Spermidine conjugate^{b} | 13.0 | 225, 295 |
| 7 | Sinapoyl malate | 15.0 | 236, 329 |
| 8 | Disinapoyl-dihexoside | 20.0 | 226, 329 |
| 9 | Kaempferol-disinapoyl- | 20.5 | 225, 268, 329 |
| | trihexoside | | |
| 11 | Disinapoyl-hexoside | 23.4 | 226, 329 |
| 12 | Trisinapoyl-dihexoside | 24.3 | 226, 326 |

^{a}UV profile could be affected by a coeluting minor component.
^{b}Structure reported by Baumert et al. (2005) Phytochemistry 66: 1334-45.

TABLE 4

| Peak | MS1 (−) m/z | MS2 (−) m/z | MS1 (+) m/z | MS2 (+) m/z |
|---|---|---|---|---|
| 1 | 1139 [M − H] | [1139] 977, | 1141 [M + H]⁺ | [1141] 531, |
| | 569 [M − 2H]²⁻ | 771, 547, | 531 | 369, 351, |
| | | 223 | | 287, 207, 175 |
| 2 | 977 [M − H]⁻ | [977] 815, | 979 [M + H]⁺ | [979] 449, |
| | | 609, 591, | | 369, 351, |
| | | 446, 284, | | 287, 207, |
| | | 205 | | 175, 127 |
| 3 | 385 [M − H]⁻ | [385] 325, | 795 | [795] 409, |
| | | 265, 247, | [2M + Na]⁺ | 247, 207, 185 |
| | | 223, 205, | | |
| | | 190, 175 | | |
| 4 | 477 [M − H]⁻ | [477] 446, | — | — |
| | | 259 | | |
| 5 | 494 [M − H]⁻ | [494] 479, | 496 [M + H]⁺ | [496] 479, |
| | | 462 | | 478, 425, |
| | | | | 408, 351, |
| | | | | 325, 283, |
| | | | | 268, 232, |
| | | | | 216, 191, |
| | | | | 175, 149 |
| 7 | 339 [M − H]⁻ | [339] 223 | 703 | [703] 363, |
| | 679 [2M − H]⁻ | [679] 339, | [2M + Na]⁺ | 229, 207, |
| | | 223 | | 175, 157 |
| 8 | 753 [M − H]⁻ | [753] 529, | 531 [M − | [531] 369, |
| | | 265, 247, | 224 + H]⁺ 777 | 351, 225, |
| | | 223, 205, | [M + Na]⁺ | 207, 175, |
| | | 190 | | 147, 127 |
| 9 | 1183 [M − H] | [591] 609, | 1185 [M + H]⁺ | [1185] 655, |
| | 591 [M − 2H]²⁻ | 652, 488, | 1207 | 369, 351, |
| | | 407, 367, | [M + Na]⁺ | 287, 207, 175 |
| | | 325, 284, | | |
| | | 265, 205, | | |
| | | 223 | | |
| 11 | 591 [M − H]⁻ | [591] 367, | 369 [M − | [369] 207, |
| | | 352, 223, | 224 + H]⁺ | 175 |
| | | 205 | | |
| 12 | 959 [M − H]⁻ | [959] 735, | 737 [M − | [737] 369, |
| | | 529, 511, | 224 + H]⁺ | 351, 207, |
| | | 247, 223, | | 175, 127 |
| | | 205 | | |

TABLE 5

| | | Rt | |
|---|---|---|---|
| Peak | Identification | (min) | UV max (nm) |
| 1 | Kaempferol-sinapoyl- | 7.4 | 222, 268, 331 |
| | tetrahexoside | | |
| 2 | Kaempferol-sinapoyl- | 7.9 | 223, 268, 331 |
| | trihexoside | | |
| 3 | Sinapoyl-hexoside | 8.5 | 235, 331 |
| 4 | Neoglucobrassicin | 11.7 | 223, 268, |
| | | | 299, 320^{a} |
| 6 | Sinapic acid^{b} | 13.7 | 227, 322 |
| 7 | Sinapoyl malate | 15.0 | 236, 329 |
| 8 | Disinapoyl-dihexoside | 20.0 | 226, 329 |
| 9 | Kaempferol-disinapoyl- | 20.5 | 225, 268, 329 |
| | trihexoside | | |
| 10 | Disinapoyl-dihexoside | 22.0 | 225, 333 |
| | (isomer of 8) | | |
| 11 | Disinapoyl-hexoside | 23.4 | 226, 329 |
| 12 | Trisinapoyl-dihexoside | 24.3 | 226, 326 |
| 13 | Trisinapoyl-dihexoside | 27.4 | 225, 326 |
| | (isomer of 12) | | |
| 14 | Methyl sinapate | 28.8 | 226, 324 |

^{a}UV profile could be affected by a coeluting minor component.
^{b}Or derivative.

TABLE 6

| Peak | MS1 (−) m/z | MS2 (−) m/z | MS1 (+) m/z | MS2 (+) m/z |
|---|---|---|---|---|
| 1 | 1139 [M − H] | [1139] 977, | 1141 [M + H]⁺ | [1141] 531, |
| | 569 [M − 2H]²⁻ | 771, 547, | 531 | 369, 351, |
| | | 223 | | 287, 207, |
| | | | | 175 |
| 2 | 977 [M − H]⁻ | [977] 815, | 979 [M + H]⁺ | [979] 449, |
| | | 609, 591, | | 369, 351, |
| | | 446, 284, | | 287, 207, |
| | | 205 | | 175, 127 |
| 3 | 385 [M − H]⁻ | [385] 325, | 795 | [795] 409, |
| | | 265, 247, | [2M + Na]⁺ | 247, 207, |
| | | 223, 205, | | 185 |
| | | 190, 175 | | |
| 4 | 477 [M − H]⁻ | [477] 446, | — | — |
| | | 259 | | |
| 6 | — | — | 225 [M + H]⁺ | — |
| | | | 247 [M + Na]⁺ | |
| | | | 207 | |
| 7 | 339 [M − H]⁻ | [339] 223 | 703 | [703] 363, |
| | 679 [2M − H]⁻ | [679] 339, | [2M + Na]⁺ | 229, 207, |
| | | 223 | | 175, 157 |
| 8 | 753 [M − H]⁻ | [753] 529, | 531 [M − | [531] 369, |
| | | 265, 247, | 224 + H]⁺ | 351, 225, |
| | | 223, 205, | 777 [M + Na]⁺ | 207, 175, |
| | | 190 | | 147, 127 |
| 9 | 1183 [M − H] | [591] 609, | 1185 [M + H]⁺ | [1185] 655, |
| | 591 [M − 2H]²⁻ | 652, 488, | 1207 | 369, 351, |
| | | 407, 367, | [M + Na]⁺ | 287, 207, |
| | | 325, 284, | | 175 |
| | | 265, 205, | | |
| | | 223 | | |
| 10 | 753 [M − H]⁻ | [753] 529, | 777 [M + Na]⁺ | [777] 553, |
| | | 265, 247, | 531 [M − | 467, 329, |
| | | 223, 205, | 224 + H]⁺ | 301, 245, |
| | | 190 | | 207, 175 |
| 11 | 591 [M − H]⁻ | [591] 367, | 369 [M − | [369] 207, |
| | | 352, 223, | 224 + H]⁺ | 175 |
| | | 205 | | |
| 12 | 959 [M − H]⁻ | [959] 735, | 737 [M − | [737] 369, |
| | | 529, 511, | 224 + H]⁺ | 351, 207, |
| | | 247, 223, | | 175, 127 |
| | | 205 | | |
| 13 | 959 [M − H]⁻ | [959] 735, | 737 [M − | [737] 369, |
| | | 511, 223, | 224 + H]⁺ | 351, 225, |
| | | 205 | | 207, 175, |
| | | | | 147,127 |
| 14 | — | — | 239 [M + H]⁺ | [239] 207, |
| | | | 261 [M + Na]⁺ | 192, 175, |
| | | | 207 | 147, 119, |
| | | | | 91 |

Many of the identified constituents are conjugates of different numbers of sinapoyl, sugar, and flavonoid moieties, eluting according to their polarity. Three flavonoid (kaempferol) derivatives bearing sinapoyl groups (peaks 1, 2, and 9) displayed a characteristic UV absorption maximum at 268 nm. Two nitrogen-containing ingredients, neoglucobrassicin (peak 4, a glucosinolate) and spermidine conjugate (peak 5, a polyamine), were also observed. Remaining structures were identified as sinapic acid derivatives. In the MS¹ spectra of these compounds, deprotonated molecules [M-H]⁻ were commonly observed in negative ion mode, whereas several positively charged species revealed a loss of 224 amu, indicating the loss of sinapic acid moiety. Similar MS² fragmentation patterns were observed for these compounds, indicating structural relationship. Due to the limitation of MS interpretation, the exact identities of the sugars and the glycosidic linkages could not be determined.

The S9-treated OE resulted in both quantitative and qualitative modifications of the metabolites. The post-treated extract displayed several differences in composition and proportions of the components, compared to the untreated OE (Tables 3-6). For example, peaks 6 (sinapic acid), 10 (a disinapoyl-dihexoside), 13 (a trisinapoyldihexoside) and 14 (methyl sinapate) appeared after S9 microsome fraction digestion, whereas the intensity of peak 7 (sinapoyl malate) was decreased. The relative intensities of flavonoid derivatives, particularly peaks 1 and 2, increased in the S9-treated OE sample. Table 7 provides a comparison of relative quantities of the identified constituents before and after S9 digestion. Clearly, sinapoyl malate (peak 7) was depleted during metabolism, whereas the kaempferol sinapoyl glycosides (peaks 1 and 2) increased.

TABLE 7

| Constituent | % Pre-Treatment | % Post-Treatment |
|---|---|---|
| Kaempferol-sinapoyl- | 5.00 | 10.27 |
| tetrahexoside | | |
| Kaempferol-sinapoyl- | 6.04 | 12.71 |
| trihexoside | | |
| Sinapoyl-hexoside | 15.84 | 15.29 |
| Neoglucobrassicin | 1.84 | 2.15 |
| Spermidine conjugate | 1.70 | 0 |
| Sinapic acid | 0 | 7.62 |
| Sinapoyl malate | 38.16 | 8.58 |
| Disinapoyl-dihexoside | 5.15 | 5.50 |
| Kaempferol-disinapoyl- | 1.12 | 1.75 |
| trihexoside | | |
| Disinapoyl-dihexoside | 0 | 1.06 |
| (isomer) | | |
| Disinapoyl-hexoside | 2.91 | 1.06 |
| Trisinapoyl-dihexoside | 9.76 | 11.67 |
| Trisinapoyl-dihexoside | 0 | 1.48 |
| (isomer) | | |
| Methyl sinapate | 0 | 7.03 |
| Other | 12.49 | 13.82 |

The percentage area under the peaks in the HPLC chromatograms was used to quantitatively compare the compositions of the pre-and post-treatment extracts.
“Other” represents other cumulative small molecules.

### Example 6: Exosome Delivery of Broccoli Extracts

Cruciferous plants such as broccoli are rich in anti-inflammatory chemicals. However, the bioavailability and stability of natural products in vivo are limited. For example, L-sulforaphane (SF) is a compound that is innately unstable and degrades easily in the physiological conditions. Even though an individual takes up a diet rich in cruciferous vegetables, a source of SF, low bioavailability and stability of SF limits the benefits of the compound. Therefore, to improve bioavailability and stability extracts of broccoli were delivered using exosomes, a natural carrier in humans.

To characterize and study the size distribution of the exosomes, DLS was performed. The average size of the exosomes was 146 nm±5 nm with a polydispersity index of 0.44±0.05, showing that the exosomes were uniformly dispersed in the solution. The cryo TEM data also confirmed the presence of saucer-shaped exosomes and their size was similar to that of the DLS data confirming the unaltered morphology of the isolated exosomes. The number of exosomes present in the sample was calculated based on a standard curve and quantification analysis indicated the presence of approximately 10⁹ exosomes/μL. The kit used in this analysis measures the activity of Acetyl-CoA Acetylcholinesterase (AChE), an enzyme that has been demonstrated to be substantially concentrated in exosomes, via an antibody-free, colorimetric test (OD 405 nm). Following this, western blot analysis was performed to check the exosome markers CD9 and HSPA8 proteins. CD9 is a protein that is typically found on the surface of the exosomes, whereas HSPA8 is found in the internal lumen of exosomes. The presence of both the surface marker and internal lumen protein reflects the unaltered biochemistry of the exosomes.

Using Eq. 1 for calculating encapsulation efficiency, the sonicated exosomes (EXO/PA-S) encapsulated 30±1.5% of the initial content of broccoli extract added, whereas the incubated exosomes (EXO/PA-I) were successful in trapping 26±6.0% of the initial amount of broccoli extract. Thus, there was no significant difference in encapsulation efficiency between the two different encapsulation methods.

To characterize and study the size distribution of the exosomes carrying broccoli extract, DLS was performed. A 1:1000 dilution of the broccoli extract-encapsulated exosomes in PBS was prepared and the size was analyzed. The average size of the EXO/PA-S exosomes was 227132 nm with a polydispersity index of 0.26±0.05. By comparison, the average size of the EXO/PA-I exosomes was 279±77 nm, with a polydispersity index of 0.23±0.05. The size of the exosomes was increased compared to the non-loaded vesicles and the polydispersity index was observed to be reduced.

The size stability the isolated exosome of nanocarriers was then assessed over a period of 40 days at −80° C. and a pH of 7.4. This analysis indicated that the size of the bare exosomes was reduced from 220 nm to approximately 197 nm by day 40. In the case of EXO/PA-S, the average size of each particle slightly shifted from 310 nm on day 0 to 267 nm on day 20 and was reduced to 169 nm on day 40. By comparison, the average size of the EXO/PA-I particles on day 0 was approximately 146 nm, which was retained on day 20, followed by a slight shift to 197 nm on day 40. Hence the results indicated that the broccoli extract-encapsulated exosomes were able to retain their structural stability over a period of 40 days.

The stability of the broccoli extract itself was assessed as well. In this study the exosomes were incubated at room temperature for 24 hours at pH 7.4, and lysed at different time points (0, 1, 15, and 24 hours). While bare broccoli extract was degraded quickly, the encapsulated broccoli extract was more stable. Approximately 25% of the bare broccoli extract was degraded in the first hour, compared to the encapsulated broccoli extract, where 75% of the broccoli extract was retained in the EXO/PA-S sample for 21 hours and 10 hours in the EXO/PA-I sample. After 24 hours, >50% of the extract was stable in EXO/PA-S.

The ability of the modified exosomes to be taken up by macrophage was analyzed using a confocal microscope. This analysis indicated that the exosomes were readily taken up by macrophages within 4 hours of incubation. The biocompatibility of broccoli extract-encapsulated the exosomes was also assessed using an Alamarblue assay. From the results, it was observed that the exosomal formulation, at a dilution of 1:50, was the most effective and non-toxic to the RAW macrophage cells (96±3.6% viability). By comparison, bare broccoli extract (1:50) reduced viability by 12±5.4% (p=0.0008) compared to the control untreated cells.

It was determined whether the broccoli extract-encapsulated exosomes possess any pro-apoptotic properties as determined by Annexin V/PI staining and flow cytometry. The live cell percentage did not significantly change across all conditions (control: 93.05±9.40% cell population; bare broccoli extract-treated cells: 88.5±13.43% cell population; EXO/PA-S-treated cells: 76±21.21% cell population; EXO/PA-I-treated cells: 93±0% cell population). In addition, no significant apoptosis was observed in either the EXO/PA-S-treated cells (16.6±16.3% cell population) or EXO/PA-I-treated cells (3.92±2.65% cell population) compared to the bare broccoli extract-treated cells (1.15±0.4% cell population) and non-treated control cells (0.19±0.16% cell population). Similarly, no significant necrosis was observed in EXO/PA-S-treated cells (6.81±9.41% cell population) or EXO/PA-I-treated cells (2.92±1.60% cell population) compared to the bare broccoli extract-treated cells (9.90±13.15% cell population) and non-treated control (6.74±9.41% cell population).

The effect of the broccoli extract-encapsulated exosomes on the expression of pro-inflammatory genes NfkB, MMP2, Cox-2, and IL-1B was also determined. Both the EXO/PA-S and EXO/PA-I formulations significantly reduced the expression of NfkB after induction of inflammation through LPS (0.009 and 0.000336, respectively, vs. 0.0026 for the LPS control; p=0.0003). For Cox-2, a significant reduction in gene expression was observed after treatment with EXO/PA-S and EXO/PA-I compared to the positive LPS control (0.000004 and 0.0001, respectively, vs 0.017 for the LPS control; p<0.0001). In the case of MMP-2, there was a notable reduction in gene expression after treatment with EXO/PA-S and EXO/PA-I compared to the LPS control (0.0000004 and 0.0000014 for EXO/PA-S and EXO/PA-I treatment, respectively, vs 0.000008 for the LPS control; p=0.002). While EXO/PA-S reduced the expression of IL-1 (0.04) compared to the LPS control (0.06), EXO/PA-I did not reduce the expression of IL-13 (0.12) compared to the LPS control (0.06) (p<0.0001).

Using a Griess assay to measure net inflammation via nitrite levels, EXO/PA-S and EXO/PA-I treatment reduced nitric oxide production 5-fold (p<0.0001) compared to LPS-activated macrophages, whereas unencapsulated broccoli extract yielded a<1 fold (p<0.001) reduction in nitric oxide. A reduction in nitric oxide production signifies a reduction in the overall inflammatory state of the LPS-activated macrophages.

Specific cytokine expression profiles were analyzed using a cytokine bead array experiment. The analysis revealed a significant reduction in TNF-x expression with EXO/PA-S (5381±50.1 pg/mL) and EXO/PA-I (4000.91±80 μg/mL) treatment (p<0.0001) compared to TNF-x expression in LPS-activated macrophages (6740.2±31.9 pg/mL). A reduction in IL-6 expression was also observed with EXO/PA-S (2423.4±42.9 pg/mL) and EXO/PA-I (1178±29.1 pg/mL) treatment (p<0.001), compared to LPS-activated macrophages (2827.4±90 μg/mL). Notably, unencapsulated broccoli extract treatment significantly reduced both TNF-α (95±4.39 pg/mL) and IL-6 (6.50±0.6 pg/mL) expression. In the case of MCP-1, expression was reduced after treatment with EXO/PA-S (2959.97±14.0 pg/mL) and EXO/PA-I (2221.5±25.74 pg/mL) compared to LPS-activated macrophages (4136±38.22 pg/mL).

The expression of pro-inflammatory (ERK) and anti-inflammatory (p-ERK, IkB) proteins in RAW macrophage cells was analyzed by western blot. β-actin and Vinculin were used as the housekeeping genes to normalize protein expression. This analysis indicated that the expression of anti-inflammatory proteins, p-ERK, and IkB increased in EXO/PA-S- and EXO/PA-I-treated cells compared to LPS control. In contrast, pro-inflammatory protein expression (ERK) was reduced in EXO/PA-S- and EXO/PA-I-treated cells. These data indicate that the immuno-suppressive potential of the broccoli extract-encapsulated exosomes is via ERK/MAPK and the IkB/NfkB signaling pathways.

### Example 7: Methods for Growing Stable Lines of Broccoli

Stable lines of broccoli plants with enhanced levels of anti-inflammatory chemicals and sulforaphane may be produced by treating broccoli seeds with a chemical mutagen (e.g., EMS); growing broccoli plant seedlings from the treated seeds; identifying broccoli plants grown from the seedlings producing the desired levels of anti-inflammatory chemicals; and propagating the identified broccoli plants to provide a stable broccoli plant line.

In an alternative approach, stable lines of broccoli plants with enhanced levels of anti-inflammatory chemicals may be produced by growing broccoli plant seedlings from the commercially-available broccoli seeds having highly pigmented seed coats; subjecting the seedlings to one or more alternating cycles of darkness, UV-C, UV-B and UV-A; subjecting the seedlings to one or more alternating cycles of temperature (0-4° C., 20° C.); identifying broccoli plants grown from the seedlings producing the desired levels of anti-inflammatory chemicals; and vegetatively propagating broccoli plants lines from the identified broccoli plants.

What is claimed is:

1. A broccoli extract comprising an enhanced level of anti-inflammatory compounds, the broccoli extract prepared by a method comprising
(a) subjecting a broccoli plant to at least one abiotic stimulus;
(b) extracting aerial material of the broccoli plant with a solvent;
(c) drying the extract;
(d) reconstituting the extract to form a concentrated broccoli extract having a concentration of about 150 mg/mL; and
(e) optionally treating the concentrated broccoli extract with an S9 liver microsome fraction,
wherein the concentrated broccoli extract comprises at least 20 weight percent sinapates and at least 30 weight percent flavonoids.

2. The broccoli extract of claim 1, wherein the broccoli plant is grown from chemically mutated seeds.

3. The broccoli extract of claim 1, wherein the at least one abiotic stimulus comprises ultraviolet light, a temperature of about 4° C., darkness, and/or blue light.

4. The broccoli extract of claim 1, wherein the concentrated broccoli extract comprises at least 30 weight percent sinapates.

5. The broccoli extract of claim 1, wherein the concentrated broccoli extract comprises at least 40 weight percent sinapates.

6. The broccoli extract of claim 1, wherein the concentrated broccoli extract comprises at least 50 weight percent flavonoids.

7. The broccoli extract of claim 6, wherein the concentrated broccoli extract comprises at least 30 weight percent sinapates.

8. The broccoli extract of claim 6, wherein the concentrated broccoli extract comprises at least 40 weight percent sinapates.

9. The broccoli extract of claim 1, wherein said extract is encapsulated in exosomes.

10. A nutraceutical product comprising the broccoli extract of claim 1.

11. A nutraceutical product comprising the broccoli extract of claim 9.

12. A nutraceutical broccoli product comprising an enhanced level of anti-inflammatory compounds, the broccoli product prepared by a method comprising subjecting a broccoli plant to at least one abiotic stimulus to enhance levels of anti-inflammatory compounds in the broccoli plant and harvesting edible portions of the broccoli plant.

13. The nutraceutical broccoli product of claim 12, wherein the at least one abiotic stimulus comprises ultraviolet light, a temperature of about 4° C., darkness, and/or blue light.

14. The nutraceutical product of claim 13, wherein said product is encapsulated in exosomes.

15. A method of treating inflammation in a subject in need thereof comprising administering to the subject an effective amount of the nutraceutical product of claim 10 to the subject thereby treating the subject's inflammation.

16. A method of treating inflammation in a subject in need thereof comprising administering to the subject an effective amount of the nutraceutical product of claim 11 to the subject thereby treating the subject's inflammation.

17. A method of treating inflammation in a subject in need thereof comprising administering to the subject an effective amount of a nutraceutical broccoli product of claim 12 thereby treating the subject's inflammation.

18. A method of treating or reducing the risk of cancer recurrence in a subject comprising administering to the subject an effective amount of the nutraceutical product of claim 10 to the subject thereby treating or reducing the risk of cancer recurrence in the subject.

19. A method of treating or reducing the risk of cancer recurrence in a subject comprising administering to the subject an effective amount of the nutraceutical product of claim 11 to the subject thereby treating or reducing the risk of cancer recurrence in the subject.

20. A method of treating or reducing the risk of cancer recurrence in a subject comprising administering to the subject an effective amount of the nutraceutical broccoli product of claim 12 to the subject thereby treating or reducing the risk of cancer recurrence in the subject.


SEQUENCE LISTING (10 sequences)

SEQ ID NO: 1 - DNA, 21 bp, synthetic construct
  source 1..21: mol_type = other DNA
aggtcggtgtgaacggatttg

SEQ ID NO: 2 - DNA, 23 bp, synthetic construct
  source 1..23: mol_type = other DNA
tgtagaccatgtagttgaggtca

SEQ ID NO: 3 - DNA, 21 bp, synthetic construct
  source 1..21: mol_type = other DNA
gagctgtgcggtctgttaaag

SEQ ID NO: 4 - DNA, 20 bp, synthetic construct
  source 1..20: mol_type = other DNA
ggtagttcggttccacacca

SEQ ID NO: 5 - DNA, 21 bp, synthetic construct
  source 1..21: mol_type = other DNA
acggtcctgaacgcatttatg

SEQ ID NO: 6 - DNA, 21 bp, synthetic construct
  source 1..21: mol_type = other DNA
ttggccccatttagcaatctg

SEQ ID NO: 7 - DNA, 19 bp, synthetic construct
  source 1..19: mol_type = other DNA
caagttccccggcgatgtc

SEQ ID NO: 8 - DNA, 21 bp, synthetic construct
  source 1..21: mol_type = other DNA
ttctggtcaaggtcacctgtc

SEQ ID NO: 9 - DNA, 22 bp, synthetic construct
  source 1..22: mol_type = other DNA
gcactacaggctccgagatgaa

SEQ ID NO: 10 - DNA, 23 bp, synthetic construct
  source 1..23: mol_type = other DNA
gtcgttggcttggttctccttgt